(19) United States
(12) Patent Application Publication
(10) Pub. No.: US 2024/0225997 A1
(43) Pub. Date: Jul. 11, 2024

(54) NATURE BASED CLEANSING COMPOSITION

(71) Applicant: L'OREAL, Paris (FR)

(72) Inventors: Hungta LIN, Teaneck, NJ (US); Changlong CHEN, Edison, NJ (US); Mariko HASEBE, New York, NY (US)

(73) Assignee: L'OREAL, Paris (FR)

(21) Appl. No.: 18/146,801

(22) Filed: Dec. 27, 2022

(51) Int. Cl. A61K 8/99; A61K 8/19; A61K 8/34; A61K 8/44; A61K 8/60; A61K 8/73; A61K 8/81; A61Q 19/10

(52) U.S. Cl. CPC A61K 8/99; A61K 8/19; A61K 8/345; A61K 8/44; A61K 8/60; A61K 8/731; A61K 8/732; A61K 8/733; A61K 8/737; A61K 8/8158; A61Q 19/10

(57) ABSTRACT

A nature-based cleansing composition includes at least one glycolipid that includes at least one rhamnolipid, at least one nature-based viscosity modifying polymeric thickener for a water-based system or at least one anionic surfactant, or a combination thereof, and water present from about 60% to about 98% by weight of the cleansing composition. The at least one nature-based viscosity modifying polymeric thickener for a water-based system may be one of algin, xanthan gum (and) sclerotium gum (and) lecithin (and) pullulan (Siligel™), xanthan gum, carbomer 980, hydroxypropyl starch phosphate, ammonium polyacryloyldimethyl taurate, hydroxyethyl cellulose, acrylate copolymer, sclerotium gum. The nature-based cleansing composition has a shelf stable thick texture and pleasing high foaming action upon application, and as compared with commercial benchmarks, provides comparable or enhanced makeup removal, including deep pore cleansing and confers cleaner skin upon removal and rinsing with a higher relative content of natural ingredients and without harsh ingredients.

### FIELD

This invention relates to a nature-based makeup removal composition.

### BACKGROUND

Removal of stubborn makeup has always been a challenge and a consumer pain point, particularly for waterproof mascara, long-wear foundation and longer-lasting lip products. Typical “make up removers” provide reasonably acceptable pigment removal capacity, but can present challenges with the removal of heavy makeups. A key drawback to existing makeup removal formulations is the inclusion of high amounts of oils which can have a greasy feel and leave skin feeling less fresh and clean than is desired by most consumers. And current makeup removers include alcohols, which can be drying to the skin, and sulfate based surfactants, silicones, and other synthetic components that can be irritating. These ingredients are less attractive to consumers seeking more natural products. Moreover, such makeup removing products do not include foaming which is a feature deemed desirable by most consumers. Current nature based cleansers often do not have pleasing aesthetics or foaming, do not apply with a thick feel, are not effective removing heavy makeup, and do not come of cleanly after rinsing.

Accordingly, there is a need for a more natural composition that will provide easy and efficacious makeup removal in a form that is thick and foamy on application and that leaves a clean and refreshing skin feel after use without the use of silicone, alcohols, harsh surfactants and sulfates.

### SUMMARY

This disclosure describes exemplary embodiments in accordance with the general inventive concepts and is not intended to limit the scope of the invention in any way. Indeed, the invention as described in the specification is broader than and unlimited by the exemplary embodiments set forth herein, and the terms used herein have their full ordinary meaning.

The disclosure provides, in various embodiments, a cleansing composition that includes a rhamnolipid and at least one nature-based viscosity modifying polymeric thickener for a water-based system. In some embodiments, the makeup removal composition may include at least one anionic surfactant. In some embodiments, the at least one anionic surfactant is selected from disodium cocoyl glutamate (and) sodium, cocoyl glutamate, or a combination thereof. In the various embodiments, water is present from about 60% to about 90% by weight of the makeup removal composition. In some embodiments, the makeup removal composition may include as the nature-based viscosity modifying polymeric thickener for a water-based system one or more of xanthan gum (and) sclerotium gum (and) lecithin (and) pullulan, xanthan gum, carbomer 980, hydroxypropyl starch phosphate, ammonium polyacryloyldimethyl taurate, hydroxyethyl cellulose, acrylate copolymer, sclerotium gum, or combinations thereof. And In some embodiments, the cleansing composition may include antimicrobial agents, pH adjusters, chelating agents, humectants, and other additives and actives. In some embodiments, the cleansing composition may include caprylyl glycol, sodium hydroxide, sorbitol, tetrasodium glutamate diacetate, glycerin, or combinations thereof.

In some embodiments, the inventive composition is 100% biodegradable and over 99% renewable, and provides unexpected benefits that include ease with overall makeup removal, particularly after rinsing, as compared with reference/comparative compositions, deep pore cleaning, and pleasing foaming. In some embodiments, the inventive composition demonstrates better performance than commercial benchmarks while including a higher amount of nature-based ingredients and lacking surfactants, alcohols and other ingredients that are perceived as harsh, such as cleanability (deep pore), makeup removability, and pleasing foaming properties as exemplified in in-vitro and in-vivo testing wherein the composition excels at removing eye and lip makeup.

In some embodiments, the cleansing composition is free or essentially free of harsh chemicals and non-nature based ingredients (silicones, alcohol, paraben, fragrance, sulfates).

In a representative embodiment, the disclosure provides a cleansing composition, comprising: at least one glycolipid comprising at least one rhamnolipid; at least one nature-based viscosity modifying polymeric thickener for a water-based system, or at least one anionic surfactant, or a combination thereof; and water present from about 60% to about 98% by weight of the cleansing composition.

In some embodiments, the at least one glycoprotein comprising rhamnolipid is sold under the name Rheance One by Evonik.

In some embodiments, the composition includes at least one nature-based viscosity modifying polymeric thickener for a water-based system, selected from algin, xanthan gum (and) sclerotium gum (and) lecithin (and) pullulan (Siligel™), xanthan gum, carbomer 980, hydroxypropyl starch phosphate, ammonium polyacryloyldimethyl taurate, hydroxyethyl cellulose, acrylate copolymer, sclerotium gum, or combinations thereof.

In some embodiments, the composition includes at least one nature-based viscosity modifying polymeric thickener for a water-based system, selected from algin, xanthan gum (and) sclerotium gum (and) lecithin (and) pullulan (Siligel™) or a combination thereof, and excluding any one or more of xanthan gum, carbomer 980, hydroxypropyl starch phosphate, ammonium polyacryloyldimethyl taurate, hydroxyethyl cellulose, acrylate copolymer, sclerotium gum, or combinations thereof.

In some embodiments, the composition includes at least one anionic surfactant.

In some embodiments, the composition includes at least one anionic surfactant selected from disodium cocoyl glutamate (and) sodium, cocoyl glutamate, or a combination thereof.

In some embodiments, the composition includes one or more of humectants, antimicrobial agents, pH adjusters, chelating agents, skin care actives, or combinations thereof, and wherein the composition is free or essentially free of one or more of silicones, alcohol, paraben, fragrance, sulfates, or combinations thereof.

In some embodiments, the composition includes one or more of glycerin, sorbitol, caprylyl glycol, sodium hydroxide, tetrasodium glutamate diacetate, or combinations thereof.

In some embodiments, the rhamnolipid is present in the cleansing composition from about 0.1% to about 20% by weight of the cleansing composition.

In some embodiments, the at least one polymeric thickener for a water-based system is present from about 0.1% to about 20% by weight, based on the weight of the cleansing composition.

In some embodiments, the rhamnolipid is present in the cleansing composition at about 5% and wherein the at least one polymeric thickener for a water-based system is present at about comprises 3.5% and comprises algin or Siligel or a combination thereof, all amounts by weight of the cleansing composition.

In some embodiments, the at least one anionic surfactant in the cleansing composition is present from about 0.01% to about 1%, or from about 0.01% to about 0.2%, or from about 0.02% to about 0.03%, all amounts by weight of the composition.

In some embodiments, the composition includes at least one humectant, present in the cleansing composition from about 1% to about 10%, and wherein water is present in the cleansing composition in a range from about 75% to about 85%, all amounts by weight of the composition.

In another representative embodiment, the disclosure provides a cleansing composition, comprising: at least one glycolipid comprising at least one rhamnolipid present in the cleansing composition from about 0.1% to about 20% by weight of the cleansing composition; (i) at least one nature-based viscosity modifying polymeric thickener for a water-based system present in the cleansing composition from about 0.1% to about 20% by weight of the cleansing composition, or (ii) at least one anionic surfactant present from about 0.01% to about 1%, the nature-based viscosity modifying polymeric thickener for a water-based system selected from algin, xanthan gum (and) sclerotium gum (and) lecithin (and) pullulan (Siligel™), xanthan gum, carbomer 980, hydroxypropyl starch phosphate, ammonium polyacryloyldimethyl taurate, hydroxyethyl cellulose, acrylate copolymer, sclerotium gum, or combinations thereof; at least one humectant present from about 1% to about 15% by weight of the cleansing composition; and water present from about 60% to about 98% by weight of the cleansing composition, wherein the composition is a makeup remover.

In some embodiments, the composition includes a nature-based viscosity modifying polymeric thickener for a water-based system comprising algin, xanthan gum (and) sclerotium gum (and) lecithin (and) pullulan (Siligel™) or a combination thereof, and excluding any one or more of xanthan gum, carbomer 980, hydroxypropyl starch phosphate, ammonium polyacryloyldimethyl taurate, hydroxyethyl cellulose, acrylate copolymer, sclerotium gum, or combinations thereof.

In some embodiments, the rhamnolipid is present in the cleansing composition at about 5% and wherein the at least one polymeric thickener for a water-based system is present at about comprises 3.5% and comprises algin or Siligel or a combination thereof, and wherein the humectant comprises glycerin and sorbitol present in combination in an amount that is about 10.5%, all amounts by weight of the cleansing composition.

In some embodiments, the composition includes one or more of antimicrobial agents, pH adjusters, chelating agents, skin care actives, or combinations thereof, and wherein the composition is free or essentially free of one or more of silicones, alcohol, paraben, fragrance, sulfates, or combinations thereof, and wherein water is present in the cleansing composition in a range from about 75% to about 85%, by weight of the composition.

In another representative embodiment, the disclosure provides a method for deep cleansing skin comprising: Providing a cleansing composition according to the disclosure, applying the composition to skin by rubbing to produce foaming, followed by rubbing to remove makeup from at least the surface of the skin, and rinsing with water to remove substantially all of the cleansing composition.

In some embodiments, the at least one glycolipid comprising at least one rhamnolipid is present in the cleansing composition from about 0.1% to about 20% by weight of the cleansing composition; the composition comprises at least one nature-based viscosity modifying polymeric thickener for a water-based system present in the cleansing composition from about 0.1% to about 20% by weight of the cleansing composition and selected from algin, xanthan gum (and) sclerotium gum (and) lecithin (and) pullulan (Siligel™), xanthan gum, carbomer 980, hydroxypropyl starch phosphate, ammonium polyacryloyldimethyl taurate, hydroxyethyl cellulose, acrylate copolymer, sclerotium gum, or combinations thereof; the composition comprises at least one humectant present from about 1% to about 15% by weight of the cleansing composition; and water is present from about 60% to about 98% by weight of the cleansing composition.

In some embodiments, the rhamnolipid is present in the cleansing composition at about 5% and wherein the at least one polymeric thickener for a water-based system is present at about comprises 3.5% and comprises algin or Siligel or a combination thereof, and wherein the humectant comprises glycerin and sorbitol present in combination in an amount that is about 10.5%, all amounts by weight of the cleansing composition.

These and other aspects of the invention are set out in the appended claims, and described in greater detail in the detailed description of the invention.

It is to be understood that both the foregoing general description and the following detailed description are exemplary and explanatory only and are not restrictive of the disclosure.

### BRIEF DESCRIPTION OF THE DRAWINGS

Features and advantages of the general inventive concepts will become apparent from the following detailed description made with reference to the accompanying drawings.

FIG. 1 is a chart demonstrating tested features of the inventive and comparative compositions;

FIG. 2 includes photographic images showing in panel A makeup applied to bioskin, in panel B the same applied makeup after use of an inventive composition followed by rinsing, in panel C a photographic image of the analyzer and in panel 4 a graphic demonstrating the depth of visual detection of material in bioskin pore;

FIG. 3 includes photographic images showing extent of foundation removal using inventive and comparative compositions;

FIG. 4 includes photographic images showing extent of lipstick removal using inventive and comparative compositions;

FIG. 5 is a photographic image of a synthetic lash substrate;

FIG. 6 is a chart demonstrating tested features of the inventive and comparative compositions; and

FIG. 7 is a chart demonstrating tested features of the inventive and comparative compositions.

### DETAILED DESCRIPTION

“All Natural” as used herein means that all carbon atoms of intentionally added ingredients are from natural sources per ISO 16128.

“Cleanser” or “cleansing composition” as used herein means and refers to any cleansing composition utilized for application to a keratinous tissue for one or more of cleansing the skin, removal of make-up and the like.

“Cosmetically acceptable” as used herein means and refers to a carrier that is compatible with any keratinous substrate.

“Cosmetically acceptable” means a carrier that is compatible with any keratinous substrate.

As used herein with respect to the foaming property of cleansing formulations, including the inventive cleansing composition, the term “foaming” means and refers to the capacity of a composition (inventive or comparative) to develop a foam upon rubbing that would be associated with applying and gently scrubbing skin or other keratinous substrates with the formulation. Reference is made to a known testing method for establishing foam formation in vitro employing equipment that is well known in the art, namely analysis using a Krauss DFA100 ™ Dynamic Foam Analyzer, which provides a relative measure of achieved foam formation. In some examples herein, foaming is described as relative formation of foam density per unit area when compared to other tested formulations under the same conditions. In some examples, bubble count using the indicated foam analyzer is provided. Foaming Capacity, or foaming or high foaming as used herein relative to foaming on a keratinous substrate, such as facial skin, is based on a scale from low to high, and represented on a scale from 1-10.

“Free” and “devoid” and “exclude” or “excluded” each indicates that no reliably measurable excluded material, for example, an excluded volatile silicone oil or excluded surfactant or other excluded material as described herein, is present in the cleansing composition. The term “essentially free” means that, while it is preferred that no excluded material is present in the cleansing composition, it is possible to have very small amounts of the excluded material in the cleansing composition of the invention, provided that these amounts do not materially affect the advantageous properties of the cleansing composition. In particular, “essentially free” means that excluded material can be present in the cleansing composition at an amount of less than 5%, or less than 4%, or less than 3%, or less than 2%, or less than 1%, or less than 0.1% by weight, based on the total weight of the cleansing composition.

“Keratinous substrate” and “keratinous tissue” each includes but is not limited to skin, hair, and nails.

“Natural or “Nature-based” as used herein means and refers to cosmetically acceptable materials and components that are one or more of directly obtained from nature, are obtained from nature with minimal processing, and are derivatives of materials that are obtained from nature. Cleansing composition according to the instant disclosure are in some embodiments up to 99.11% natural, or “Highly natural.”

“Pore cleansing” refers to the removal of makeup and other material from within pores of keratinous material. The examples herein provide evidence of pore cleansing with embodiments of the inventive composition.

“Petrochemical-free” means that excluded petrochemical have not been added as a component. Some specific but non-limiting examples of petrochemicals that are lacking from the cosmetic cleansing composition include benzalkonium chloride, isododecane, isohexadecane and the like. In some embodiments, the cleansing composition is not free from petrochemicals, but is formulated to have an amount of petrochemicals that is at or below the amounts found in similar compositions that are commercially available.

“Silicone-free” means that excluded silicones have not been added as a component. In some embodiments, a composition is devoid of silicones. Some specific but non-limiting examples of silicones that are lacking from the cosmetic cleansing composition includes, but is not limited to, silicone polymers, for example selected from dimethicone, cyclopentasiloxane, and other silicone oils, and silicone elastomers.

“Skin” as used herein means and refers to skin materials containing keratin such as facial and body skin, scalp, eyebrows, and lips.

“Stable” and “Stability” as used herein means that the foaming cleansing composition does not show any signs of significant changes in one or more of color, odor, pH, or viscosity at room temperature over at least a two-month period as evidenced by one or both of unaided visual or microscopic inspection wherein the foaming cleansing composition is free or substantially free from formation of crystals, precipitates, or phase separation of oily and water components. In some embodiments, the inventive composition, including as exemplified herein, is developed as a thick, creamy formulation for packaging and use in a tube container, such that a measure of stability is retention of the composition in the tube without runniness for at least one month, or two months, or more at ambient temperatures and at temperatures in the range from about 25° C. to about 45° C. In various embodiments, an inventive composition remains stable at temperatures in the range from about 25° C. to about 45° C., over a time period of at least one month, or at least two months, or longer, or any value, range, or sub-range therebetween. In some embodiments, a foaming cleansing composition is stable at 25° C. for at least one month, or at least two months, or longer, or any value, range, or sub-range therebetween. In some embodiments, a foaming cleansing composition is stable at 45° C. for at least one month, or at least two months, or longer, or any value, range, or sub-range therebetween.

“Surfactant-free” means that excluded surfactants have not been added as a component. In some particular embodiments, the cleansing composition is devoid of surfactants. In some embodiments, the cleansing composition is devoid of ethylene glycol (PEG) based surfactants or of sulfate/sulfonate based surfactant, or combinations thereof. Those of skill in the art will appreciate that a surfactant may be present in a composition via its presence in one or more of the formulation components; thus, in some embodiments the cleansing composition may be “essentially surfactant-free” wherein surfactant is present at a concentration that does not exceed 5% by weight, and in some instances is present not more than 3% by weight, and in some instances is present not more than 1% by weight, based on the weight of the cosmetic cleansing composition. In some particular embodiments “surfactant-free” means that the cosmetic cleansing composition is free or devoid specifically of surfactant. Some specific but non-limiting examples of surfactants that are lacking from the cleansing composition includes those with a C10-C20 fatty alcohol or acid hydrophobe condensed with from about 2 to about 100 moles of ethylene oxide or propylene oxide per mole of hydrophobe; C2-C10 alkyl phenols condensed with from 2 to 20 moles of alkylene oxide; mono- and di-fatty acid esters of ethylene glycol; fatty acid monoglyceride; sorbitan; mono- and di- C8-C20 fatty acids; polyoxyethylene sorbitan; alkyl polyglycosides and saccharide fatty amides (e.g. methyl gluconamides); alkyl ether sulfate and sulfonates; alkyl sulfates and sulfonates; alkylbenzene sulfonates; alkyl and dialkyl sulfosuccinates; C8-C20 acyl isethionates; C8-C20 alkyl ether phosphates; alkylethercarboxylate. Some specific surfactants that are lacking from the cosmetic cleansing composition include PEG-100 Stearate; PEG-20 Stearate and other esters of Poly(Ethylene Glycol); Sucrose Stearate and other emulsifiers based on sugar esters; Glyceryl Stearate and other glycerol esters; Disodium Ethylene Dicocamide PEG-15 Disulfate; Sodium Steroyl Glutamate and other fatty amides; Steareth-100 and other fatty ethers; Acrylates/C10-30 Alkyl Acrylate Crosspolymer and similar polymeric emulsifiers.

In accordance with the various embodiments according to the disclosure, the cleansing composition includes a rhamnolipid and at least one nature-based viscosity modifying polymeric thickener for a water-based system. In some embodiments, the cleansing composition may include at least one anionic surfactant. In some embodiments, the at least one anionic surfactant is selected from disodium cocoyl glutamate (and) sodium, cocoyl glutamate, or a combination thereof. In the various embodiments, water is present from about 60% to about 90% by weight of the cleansing composition. In some embodiments, the cleansing composition may include as the nature-based viscosity modifying polymeric thickener for a water-based system one or more of xanthan gum (and) sclerotium gum (and) lecithin (and) pullulan, xanthan gum, carbomer 980, hydroxypropyl starch phosphate, ammonium polyacryloyldimethyl taurate, hydroxyethyl cellulose, acrylate copolymer, sclerotium gum, or combinations thereof. And In some embodiments, the cleansing composition may include antimicrobial agents, pH adjusters, chelating agents, humectants, and other additives and actives. In some embodiments, the cleansing composition may include caprylyl glycol, sodium hydroxide, sorbitol, tetrasodium glutamate diacetate, glycerin, or combinations thereof.

According to the disclosure, the inventive composition is 100% biodegradable and over 99% renewable, and provides unexpected benefits that include ease with overall makeup removal, particularly after rinsing, as compared with reference/comparative compositions, deep pore cleaning, and pleasing foaming. Typical single bio-surfactant water based compositions show very low foaming performance. The inventive cosmetic composition that includes a bio-surfactant rhamnolipid (Glycolipid) together with at least one natural polymer thickener, as exemplified herein, including algin/alginate and Siligel™, demonstrates better performance than commercial benchmarks, such as cleanability (deep pore), makeup removability, and pleasing foaming properties as exemplified in in-vitro and in-vivo testing. Thus, the cleansing composition provides higher surface activity and biodegradability, lower toxicity, and environmental compatibility with a pleasing aesthetic including pleasing foaming and effective makeup removal.

In some embodiments, the cleansing composition is free or essentially free of harsh chemicals and non-nature based ingredients (silicones, alcohol, paraben, fragrance, sulfates).

Through expert evaluations of makeup removal efficacy, the cleansing composition according to the disclosure has demonstrated performance on par with or better than competitor compositions when removing foundation and excels at removing eye and lip makeup.

In some embodiments, the cleansing composition is a skin cleanser, shampoo, or body wash and in some particular embodiments, the composition is a makeup remover.

### Rhamnolipids:

In accordance with the disclosure, the cleansing composition includes at least one glycolipid selected from rhamnolipids.

The cleansing composition according to the invention comprises one or more rhamnolipids.

Rhamnolipids are glycolipids produced by various bacterial species. They consist of one rhamnose fragment (mono-rhamnolipid) or of two rhamnose fragments (di-rhamnolipid) linked by a glycosidic bond to one, two or three chains of β-hydroxylated fatty acids linked to one another by an ester bond.

More specifically, these mono-rhamnolipids and di-rhamnolipids correspond to the following formula (I):

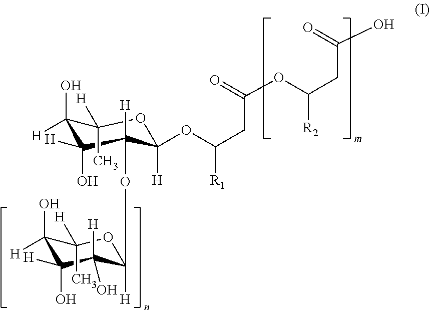

in which:
   m denotes an integer equal to 2, 1 or 0,
   n denotes an integer equal to 1 or 0, and
   R₁ and R₂, each independently represent identical or different hydrocarbon radicals having from 2 to 24 carbon atoms, from 5 to 13 carbon atoms, that are branched or unbranched, substituted or unsubstituted, in particular hydroxy-substituted, and saturated or unsaturated, a singly, doubly or triply unsaturated alkyl radical. Thus, when n is equal to 0, the formula (I) protects mono-rhamnolipids and, when n is equal to 1, it protects di-rhamnolipids.

The cleansing composition according to the invention comprises at least one di-rhamnolipid.

The cleansing composition according to the invention comprises at least one di-rhamnolipid of formula (I) in which:
m denotes an integer equal to 2, 1 or 0;
n denotes an integer equal to 1; and
R₁ and R₂, each independently represent identical or different hydrocarbon radicals having from 2 to 24 carbon atoms, from 5 to 13 carbon atoms, that are branched or unbranched, substituted or unsubstituted, in particular hydroxy-substituted, and saturated or unsaturated, a singly, doubly or triply unsaturated alkyl radical, and also the salts thereof, solvates thereof and optical isomers thereof.

The glycosidic bond between the two rhamnose fragments may be in the alpha or beta configuration and is in the alpha configuration.

In the context of the invention,
the salts of the di-rhamnolipids of formula (I) are more particularly the carboxylate salts thereof with an organic or inorganic cation and especially with a cation selected from sodium, potassium, calcium and ammonium.
the solvated forms of the di-rhamnolipids of formula (I) are more particularly those solvated with one or more molecules of water or of organic solvents, for example a hydrate or a solvate of a linear or branched alcohol, such as ethanol or isopropanol, the optically active carbon atoms of the fatty acids being in the form of the R enantiomers, and
the term “alkyl” radical denotes a saturated, linear or branched aliphatic group; for example, a C₁-C₂₀ alkyl group having a linear or branched hydrocarbon chain of 1 to 20 carbon atoms, more particularly a methyl, ethyl, propyl, isopropyl, butyl, isobutyl, tert-butyl, pentyl, hexyl, heptyl, octyl, nonyl, decyl, undecyl, dodecyl, tridecyl, tetradecyl, pentadecyl, hexadecyl, heptadecyl, octadecyl, nonadecyl or eicosyl.

The cleansing composition according to the invention comprises at least one di-rhamnolipid of formula (I) in which:
m denotes an integer equal to 2, 1 or 0;
n denotes an integer equal to 1; and
R₁ and R₂, which are identical or different, are selected from pentenyl, hexenyl, heptenyl, octenyl, nonenyl, decenyl, undecenyl, dodecenyl and tridecenyl radicals and radicals of formula —(CH₂)ₒCH₃, with o denoting an integer ranging from 1 to 23, in particular from 3 to 15 and more particularly from 4 to 12.

According to one embodiment of the invention, the cleansing composition according to the invention comprises at least one di-rhamnolipids of general formula (I) in which m is equal to 1.

According to one embodiment of the invention, the cleansing composition according to the invention comprises a mixture of at least two, at least three, di-rhamnolipids of general formula (I) in which m is equal to 1.

According to another embodiment of the invention, the cleansing composition according to the invention comprises a mixture comprising at least one mono-rhamnolipid.

More, the cleansing composition according to the invention comprises at least one dirhamnolipid of the following formula (II):

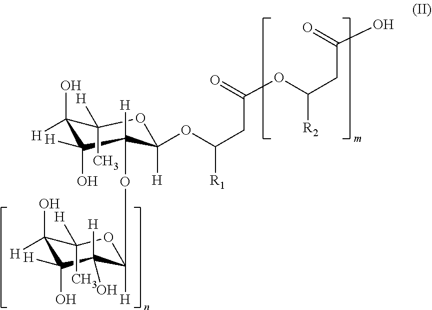

in which:
m denotes an integer equal to 2, 1 or 0; m is equal to 1,
n denotes an integer equal to 1,
R₁ is a —(CH₂)ₚ—CH₃ radical, with p being an integer varying from 1 to 23, from 4 to 12,
R₂ is a —(CH₂)_{q}—CH₃ radical, with q being an integer varying from 1 to 23, from 4 to 12, and also the salts thereof, solvates thereof and optical isomers thereof.

By way of illustration and without limiting the di-rhamnolipids of formula (II) that may be suitable for the invention, mention may be made in particular of the compounds of formula di-RL-CXCY, such as are defined in Table 1 below.

The formula di-RL-CXCY is an alternative way of writing in order to represent a di-rhamnolipid (di-RL) functionalized by two radicals R₁ and R₂ respectively represented by the symbols CX and CY, the integers X and Y being respectively equal to p+4 and q+4.

TABLE 1
Di-Rhamnolipids

| Composes | Di-RL-CXCY | p | q |
|---|---|---|---|
| 1 | diRL-C8C8 | 4 | 4 |
| 2 | diRL-C8C10 | 4 | 6 |
| 3 | diRL C10C8 | 6 | 4 |
| 4 | diRL-C10C10 | 6 | 6 |
| 5 | diRL-C10C12 | 6 | 8 |
| 6 | diRL-C12C10 | 8 | 6 |
| 7 | diRL-C12C12 | 8 | 8 |
| 8 | diRL-C12C14 | 8 | 10 |
| 9 | diRL-C14C12 | 10 | 8 |
| 10 | diRL-C14C14 | 10 | 10 |
| 11 | diRL-C14C16 | 10 | 12 |
| 12 | diRL-C16C14 | 12 | 10 |
| 13 | diRL-C16C16 | 12 | 12 |

According to an embodiment, the cleansing composition according to the invention comprises at least one di-rhamnolipid of formula (II) in which p and q are identical and equal to 6 and m is equal to 1, also referred to as di-RL-C10C10, or one of the salts, solvates and optical isomers thereof.

In some embodiments, the di-rhamnolipid of formula (II) in which p and q are identical and equal to 6 and m is equal to 1 is present in the cleansing composition according to the invention in a proportion of at least 50% by weight and of from 51% to 85% by weight, relative to the total weight of rhamnolipids.

According to another embodiment, the cleansing composition according to the invention comprises at least one di-rhamnolipid of formula (II) in which m is equal to 1, p is equal to 6 and q is equal to 8.

According to another embodiment, the cleansing composition according to the invention comprises at least one di-rhamnolipid of formula (I) in which n and m are equal to 1, R₁ represents a —(CH₂)ₒCH₃ radical, with o being an integer varying from 4 to 12, and R₂ is selected from the pentenyl, hexenyl, heptenyl, octenyl, nonenyl, decenyl, undecenyl, dodecenyl and tridecenyl radicals; R₁ represents a —(CH₂)₆CH₃ radical and R₂ a nonenyl radical.

According to another preferred embodiment, the cleansing composition according to the invention comprises a mixture of at least two, in particular at least three, di-rhamnolipids of formula (I) or of formula (II) selected from:
a di-rhamnolipid of formula (II) in which p and q are identical and equal to 6 and m is equal to 1;
a di-rhamnolipid of formula (II) in which m is equal to 1, p is equal to 6 and q is equal to 8; and
at least one di-rhamnolipid of formula (VI) in which n and m are equal to 1, R₁ represents a —(CH₂)ₒCH₃ radical, with o being an integer varying from 4 to 12, and R₂ is selected from the pentenyl, hexenyl, heptenyl, octenyl, nonenyl, decenyl, undecenyl, dodecenyl and tridecenyl radicals; R₁ represents a —(CH₂)₆CH₃ radical and R₂ a nonenyl radical.

In some embodiments, the cleansing composition according to the invention comprises a mixture of at least two, in particular at least three, di-rhamnolipids of formula (I) or of formula (II) selected from:
1 at least 50% by weight and of from 51% to 85% by weight of a di-rhamnolipid of formula (II) in which p and q are identical and equal to 6 and m is equal to 1, relative to the total weight of rhamnolipids
from 0.5% to 25% by weight, from 5% to 15% by weight, of a dirhamnolipid of formula (II) in which p is equal to 6, q is equal to 8 and m is equal to 1, relative to the total weight of rhamnolipids, and
from 0.5% to 15% by weight, from 3% to 12% by weight, from 5% to 10% by weight, of a dirhamnolipid of formula (VI) in which n and m are equal to 1, R₁ represents a —(CH₂)₆CH₃ radical and R₂ represents a nonenyl radical, relative to the total weight of rhamnolipids.

As specified above, rhamnolipids are customarily prepared by processes known to those skilled in the art starting from bacterial producers, such as Pseudomonas.

Appropriate fermentation methods are reviewed by D. Haferburg, R. Hommel, R. Claus and H. P. Kleber in Adv. Biochem. Ing./Biotechnol. (1986), 33, 53-90, and by F. Wagner, H. Bock and A. Kretschmar in Fermentation (ed. R. M. Lafferty) (1981), 181-192, Springer Verlag, Vienna.

Use may be made, as rhamnolipid, of the one sold under the name Rheance One by Evonik (INCI name: glycolipids).

In some embodiments the rhamnolipid is present in the cleansing composition at about 5% by weight of the cleansing composition.

The at least one rhamnolipid glycolipid is present in the cleansing composition according to the disclosure from about 0.1% to about 20% by weight, or from about 1% to about 10% by weight, or from about 2% to about 8%, or from about 3% to about 5%, or about 5%, or any suitable combination, sub-combination, range, or sub-range thereof by weight, based on the weight of the cleansing composition.

Thus, one or a combination of rhamnolipid glycolipid may be present, by weight, based on the total weight of the cleansing composition, from about 0.1, 0.2, 0.3, 0.4, 0.5, 0.6, 0.7, 0.8, 0.9, 1.0, 2, 3, 4, 5, 6, 7, 8, 9, 10, 11, 12, 13, 14, 15, 16, 17, 18, 19 to about 20 weight percent, including increments and ranges therein and there between.

### (ii) Viscosity Modifying Polymer

In various embodiments, the cleansing composition may include at least one nature-based viscosity modifying polymer, in particular a nature-based viscosity modifying polymer for a water-based system. In various embodiments, the viscosity modifying polymer may exclude cationic polymers and may specifically exclude chitosan (oligomeric, polymeric, derivatives).

In some embodiments, the cleansing composition includes nature-based viscosity modifying polymers and instead includes at least one anionic surfactant. In some embodiments, the at least one nature-based viscosity modifying polymer is in addition to at least one anionic surfactant. In some embodiments, the cleansing composition includes at least one nature-based viscosity modifying polymer and at least one anionic surfactant. In some embodiments, the cleansing composition excludes added anionic surfactants.

In some embodiments, the nature-based viscosity modifying polymer for a water-based system may be selected from the group consisting of nonionic, anionic, cationic, and amphoteric polymers. In some embodiments the viscosity modifying polymer is selected from polysaccharides that include alginates/algin, chitosan, chitin, starches, celluloses, galactomannans such as guar gums and its derivatives, including hydroxypropyl guar, cationic guar derivatives, gums of microbial origin, including xanthan gum, scleroglucan gum, mucopolysaccharides, chondroitin sulfates, and mixtures thereof, and gums derived from plant exudates, including locust bean gums, gum arabic, ghatti gum, karaya gum, gum tragacanth, carrageenan gum, agar gum and carob gum, cellulose-based polymers, including methylcellulose, hydroxyalkylcellulose, ethylhydroxyethylcelluloses, hydroxyethylcellulose, hydroxypropylcellulose, carboxymethylcellulose, cationic cellulose ether derivatives, quaternized cellulose derivatives, nitrocellulose, hemicellulose and hemicellulose derivatives, mannans, xylans, lignins, arabans, galacturonans, alginate-based compounds, glucuronoxylans, arabinoxylans, xyloglucans, glucomannans, fructosans such as inulin, pectic acids and pectins, arabinogalactans, agars, glycosaminoglycans, gelatin, gellan, crosslinked homopolymers of acrylic acid, and combinations thereof.

In some particular embodiments, the cleansing composition comprises at least one viscosity modifying polymer selected from the group consisting of algin, xanthan gum (and) sclerotium gum (and) lecithin (and) pullulan (Siligel™), xanthan gum, carbomer 980, hydroxypropyl starch phosphate, ammonium polyacryloyldimethyl taurate, hydroxyethyl cellulose, acrylate copolymer, sclerotium gum, wherein the viscosity modifying polymer is separate from and in addition to the cationic polymer and wherein the viscosity modifying polymer excludes cationic polymers and specifically excludes chitosan.

In some embodiments, the cleansing composition excludes one or more of xanthan gum, carbomer 980, hydroxypropyl starch phosphate, ammonium polyacryloyldimethyl taurate, hydroxyethyl cellulose, acrylate copolymer, sclerotium gum, or combinations thereof.

In some embodiments, the at least one viscosity modifying polymer for a water-based system is present from about 0.1% to about 20% by weight, based on the weight of the cleansing composition and comprises algin or Siligel or a combination thereof.

The at least one viscosity modifying polymer present in the cleansing composition according to the disclosure from about 0.1% to about 20% by weight, or from about 0.5% to about 10% by weight, or from about 1% to about 5%, or from about 1.5% to about 4.5%, or about 3.5%, or any suitable combination, sub-combination, range, or sub-range thereof by weight, based on the weight of the cleansing composition.

Thus, one or a combination of polymers may be present, by weight, based on the total weight of the cleansing composition, from about 0.1, 0.2, 0.3, 0.4, 0.5, 0.6, 0.7, 0.8, 0.9, 1.0, 2, 3, 4, 5, 6, 7, 8, 9, 10, 11, 12, 13, 14, 15, 16, 17, 18, 19 to about 20 weight percent, including increments and ranges therein and there between.

(iii) Anionic Surfactants

In accordance with the various embodiments, the cleansing composition may include at least one anionic surfactant. In some embodiments, the at least one anionic surfactant may be selected from disodium cocoyl glutamate (and) sodium, cocoyl glutamate, or a combination thereof. In some embodiments, the at least one anionic surfactant is in addition to the at least one viscosity modifying polymer. In some embodiments, the at least one anionic surfactant is an alternative to the at least one viscosity modifying polymer, such that the cleansing composition excludes a nature-based viscosity modifying polymer.

In some embodiments, each of the at least one anionic surfactant in the cleansing composition is present from about 0.01% to about 1%, or from about 0.01% to about 0.2%, or from about 0.02% to about 0.03%, or any suitable combination, sub-combination, range, or sub-range thereof by weight, based on the weight of the cleansing composition. One of ordinary skill in the art, however, will appreciate that other ranges are within the scope of the invention.

Thus, when present, each of the at least one anionic surfactant is present by weight, based on the total weight of the cleansing composition, from about 0.01, 0.015, 0.02, 0.025, 0.03, 0.035, 0.04, 0.045, 0.05, 0.055, 0.06, 0.065, 0.07, 0.075, 0.08, 0.085, 0.09, 0.095, 0.1, 0.2, 0.3, 0.4, 0.5, 0.6, 0.7, 0.8, 0.9, to about 1.0 percent, including increments and ranges therein and there between.

More generally, at least one anionic surfactant, if present, may be selected from non-sulfate anionic surfactants, sulfate anionic surfactants, their salts thereof, or mixtures thereof.

### Non-Sulfate Anionic Surfactants

In some instances, the non-sulfate anionic surfactant(s) are the predominant type of surfactant in the surfactant system (i.e., there is a higher percentage of non-sulfate anionic surfactant(s) than any other single surfactant type in the cleansing composition). Moreover, in some instances, the total amount of non-sulfate anionic surfactants in the surfactant system is higher than the total amount of all other surfactant types in the surfactant system including amphoteric surfactants, nonionic surfactants, and any sulfate-based anionic surfactant that may be present. In other words, the phrase “all other surfactants” means any and all surfactants in the cleansing composition other than anionic surfactants.

When present, the total amount of non-sulfate anionic surfactants in the foaming cleansing compositions can vary but typically ranges from about 4% to about 30%, based on the total weight of the foaming cleansing composition. In some cases, the total amount of non-sulfate anionic surfactants in the foaming cleansing composition may be from about 4% to about 25%, or from about 5% to about 25%, or from about 5% to about 23%, or from about 6% to about 24%, or from about 6% to about 25%, or from about 7% to about 25%, or from about 8% to about 25%, or from about 10% to about 23%, %, or any suitable combination, sub-combination, range, or sub-range thereof by weight, based on the weight of the foaming cleansing composition based.

In various embodiments, the total amount of non-sulfate anionic surfactants in the foaming cleansing compositions, if present, is typically at about 6, 6.5, 7, 7.5, 8, 8.5, 8.8, 9, 9.5, 10, 10.5, 11, 11.4, 12, 12.5, 13, 13.5, 14, 14.5, 15, 15.5, 16, 16.5, 17, 17.5, 18, 18.5, 19, 19.5, 20, 20.5, 21, 21.5, 22, 22.5, 23, 23.5, 24, 24.5, to about 25, weight percent based on the total weight of the foaming cleansing composition, including increments and ranges therein and there between.

Useful non-sulfate anionic surfactants include, but are not limited to, alkyl sulfonates, alkyl sulfosuccinates, alkyl sulfoacetates, acyl isethionates, alkoxylated monoacids, acyl amino acids such as acyl taurates, acyl glycinates, acyl glutamates, acyl sarcosinates, salts thereof, or mixtures thereof. Non-limiting examples of useful non-sulfate anionic surfactants are provided below.

### Acyl Isethionates

Non-limiting examples of useful acyl isethionates and their salts include those of formula (III) and (IV):

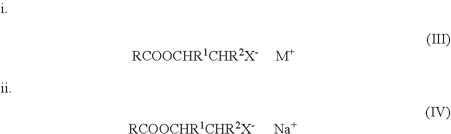

wherein:
R, R¹, and R² are each independently chosen from H or an alkyl chain having 1-24 carbon atoms, said chain being saturated or unsaturated, linear or branched;
X is COO⁻ or SO₃⁻; and
M is any suitable cation.

Although the cation may be chosen from any suitable cation as in formula (III), including, for example, alkali metal ion such as sodium or potassium, ammonium ions, or alkanolammonium ions such as monoethanolammonium or triethanolammonium ions, sodium is an cation, as in formula (IV). In various embodiments, RCO—represents the coconut acid moiety. Non-limiting examples of acyl isethionates include sodium cocoyl isethionate, sodium lauroyl isethionate, sodium lauroyl methyl isethionate, and sodium cocoyl methyl isethionate.

### Acyl Sarcosinates

Non-limiting examples of acyl sarcosinates and their salts include potassium lauroyl sarcosinate, potassium cocoyl sarcosinate, sodium cocoyl sarcosinate, sodium lauroyl sarcosinate, sodium myristoyl sarcosinate, sodium oleoyl sarcosinate, sodium palmitoyl sarcosinate, and ammonium lauroyl sarcosinate.

In some embodiments, the total amount of acyl sarcosinates in the cleansing composition, if present, is from about 0.5 to about 10%, all amounts by weight based on the weight of the composition. In some instance, the total amount of acyl sarcosinates in the cleansing composition is from about 0.5 to about 10%, about 1 to about 8%, about 1.5 to about 7%, about 1.75 to about 6%, 2 to about 5%, about 2 to about 4%, about 2.5 to about 4%, about 2.5 to about 3.5%, or about 2.7 to about 3%, all amounts by weight based on the weight of the composition, including all ranges and subranges therebetween.

### Alkyl Sulfonates

Useful alkyl sulfonates and their salts include alkyl aryl sulfonates, primary alkane disulfonates, alkene sulfonates, hydroxyalkane sulfonates, alkyl glyceryl ether sulfonates, alpha-olefinsulfonates, sulfonates of alkylphenolpolyglycol ethers, alkylbenzenesulfonates, phenvlalkanesulfonates, alpha-olefinsulfonates, olefin sulfonates, alkene sulfonates, hydroxyalkanesulfonates and disulfonates, secondary alkanesulfonates, paraffin sulfonates, ester sulfonates, sulfonated fatty acid glycerol esters, and alpha-sulfo fatty acid methyl esters including methyl ester sulfonate.

In some instances, an alkyl sulfonate of formula (V) is particularly useful:

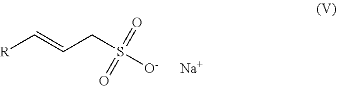

wherein R is selected from H or alkyl chain that has 1-24 carbon atoms, 6-24 carbon atoms, more, 8 to 20 carbon atoms, said chain being saturated or unsaturated, linear or branched, substituted or unsubstituted. Sodium is shown as the cation in the above formula (V) but the cation may be an alkali metal ion such as sodium or potassium, ammonium ions, or alkanolammonium ions such as monoethanolammonium or triethanolammonium ions.

In some instances, the alkyl sulfonate(s) are selected from C₈-C₁₆ alkyl benzene sulfonates, C₁₀-C₂₀ paraffin sulfonates, C₁₀-C₂₄ olefin sulfonates, salts thereof, or mixtures thereof. A non-limiting but particularly useful example of a C₁₀-C₂₄ olefin sulfonate that can be used in the instant compositions is sodium C₁₄-16 olefin sulfonate.

### Alkyl Sulfosuccinates

Non-limiting examples of useful alkyl sulfosuccinates and their salts include those of formula (VI):

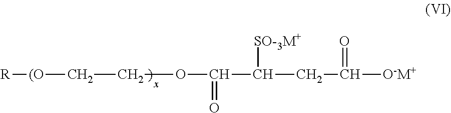

wherein:
1. R is a straight or branched chain alkyl or alkenyl group having 10 to 22 carbon atoms, 10 to 20 carbon atoms;
2. x is a number that represents the average degree of ethoxylation, and can range from 0 to about 5, from 0 to about 4, and most from about 2 to about 3.5; and
3. M, which can be the same or different, is chosen from any suitable monovalent cation.

Preferred cations are alkali metal ions such as sodium or potassium, ammonium ions, or alkanolammonium ions such as monoethanolammonium or triethanol-ammonium ions.

Non-limiting examples of alkyl sulfosuccinates salts include disodium oleamido MIPA sulfosuccinate, disodium oleamido MEA sulfosuccinate, disodium lauryl sulfosuccinate, disodium laureth sulfosuccinate, diammonium lauryl sulfosuccinate, diammonium laureth sulfosuccinate, dioctyl sodium sulfosuccinate, disodium oleamide MEA sulfosuccinate, sodium dialkyl sulfosuccinate, or mixtures thereof.

### Alkyl Sulfoacetates

Non-limiting examples of alkyl sulfoacetates and their salts include, for example, alkyl sulfoacetates such as C4-C18 fatty alcohol sulfoacetates and/or salts thereof, for example, sodium lauryl sulfoacetate. Useful cations for the salts include alkali metal ions such as sodium or potassium, ammonium ions, or alkanolammonium ions such as monoethanolammonium or triethanolammonium ions.

### Alkoxylated Monoacids

Non-limiting examples of alkoxylated monoacids include compounds corresponding to formula (VII):

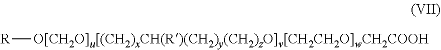

wherein:
1. R is a hydrocarbon radical containing from about 6 to about 40 carbon atoms;
2. R′ represents hydrogen or alkyl;
3. u, v, and w, which may be identical or different, independently represent numbers from 0 to 60;
4. x, y, and z, which may be identical or different, independently represent numbers from 0 to 13; and
5. the sum of x+y+z>0.

Compounds corresponding to formula (VII) can be obtained by alkoxylation of alcohols R—OH with ethylene oxide as the sole alkoxide or with several alkoxides and subsequent oxidation. The numbers u, v, and w each represent the degree of alkoxylation. Whereas, on a molecular level, the numbers u, v, and w and the total degree of alkoxylation can only be integers, including zero, on a macroscopic level they are mean values in the form of broken numbers.

In formula (VII), R is linear or branched, acyclic or cyclic, saturated or unsaturated, aliphatic or aromatic, substituted or unsubstituted. Typically, R is a linear or branched, acyclic C6-C40 alkyl or alkenyl group or a C1-C40 alkyl phenyl group, more typically a C8-C22 alkyl or alkenyl group, or a C4-C18 alkyl phenyl group, and even more typically a C12-C18 alkyl group or alkenyl group or a C6-C16 alkyl phenyl group. Further, u, v, w, independently of one another, are typically chosen from a number ranging from 2 to 20, more typically a number ranging from 3 to 17, and most typically a number ranging from 5 to 15. Further still, x, y, z, independently of one another, are typically chosen from a number ranging from 0 to 13, more typically a number ranging from 1 to 10, and most typically a number ranging from 2 to 8.

Suitable alkoxylated monoacids include, but are not limited to: Butoxynol-5 Carboxylic Acid, Butoxynol-19 Carboxylic Acid, Capryleth-4 Carboxylic Acid, Capryleth-6 Carboxylic Acid, Capryleth-9 Carboxylic Acid, Ceteareth-25 Carboxylic Acid, Coceth-7 Carboxylic Acid, C9-11 Pareth-6 Carboxylic Acid, C11-15 Pareth-7 Carboxylic Acid, C12-13 Pareth-5 Carboxylic Acid, C12-13 Pareth-8 Carboxylic Acid, C12-13 Pareth-12 Carboxylic Acid, C12-15 Pareth-7 Carboxylic Acid, C12-15 Pareth-8 Carboxylic Acid, C14-15 Pareth-8 Carboxylic Acid, Deceth-7 Carboxylic Acid, Laureth-3 Carboxylic Acid, Laureth-4 Carboxylic Acid, Laureth-5 Carboxylic Acid, Laureth-6 Carboxylic Acid, Laureth-8 Carboxylic Acid, Laureth-10 Carboxylic Acid, Laureth-11 Carboxylic Acid, Laureth-12 Carboxylic Acid, Laureth-13 Carboxylic Acid, Laureth-14 Carboxylic Acid, Laureth-17 Carboxylic Acid, PPG-6-Laureth-6 Carboxylic Acid, PPG-8-Steareth-7 Carboxylic Acid, Myreth-3 Carboxylic Acid, Myreth-5 Carboxylic Acid, Nonoxynol-5 Carboxylic Acid, Nonoxynol-8 Carboxylic Acid, Nonoxynol-10 Carboxylic Acid, Octeth-3 Carboxylic Acid, Octoxynol-20 Carboxylic Acid, Oleth-3 Carboxylic Acid, Oleth-6 Carboxylic Acid, Oleth-10 Carboxylic Acid, PPG-3-Deceth-2 Carboxylic Acid, Capryleth-2 Carboxylic Acid, Ceteth-13 Carboxylic Acid, Deceth-2 Carboxylic Acid, Hexeth-4 Carboxylic Acid, Isosteareth-6 Carboxylic Acid, Isosteareth-11 Carboxylic Acid, Trudeceth-3 Carboxylic Acid, Trideceth-6 Carboxylic Acid, Trideceth-8 Carboxylic Acid, Trideceth-12 Carboxylic Acid, Trideceth-3 Carboxylic Acid, Trideceth-4 Carboxylic Acid, Trideceth-7 Carboxylic Acid, Trideceth-15 Carboxylic Acid, Trideceth-19 Carboxylic Acid, Undeceth-5 Carboxylic Acid, or mixtures thereof.

### Acyl Amino Acids

Acyl amino acids that may be used include, but are not limited to, amino acid surfactants based on alanine, arginine, aspartic acid, glutamic acid, glycine, isoleucine, leucine, lysine, phenylalanine, serine, tyrosine, valine, sarcosine, threonine, and taurine. The most common cation associated with the acyl amino acid can be sodium or potassium. Alternatively, the cation can be an organic salt such as triethanolamine (TEA) or a metal salt.

Non-limiting examples of useful acyl amino acids include those of formula (VIII):

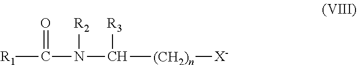

wherein:
R¹, R², and R³ are each independently selected from H or an alkyl chain having 1-24 carbon atoms, said chain being saturated or unsaturated, linear or branched, substituted or unsubstituted;
n ranges from 0 to 30; and
X is COO⁻ or SO₃⁻.

In various embodiments, RCO—represents the coconut acid moiety. A non-limiting example is sodium cocoyl glycinate.

### Acyl Taurates

Non-limiting examples of acyl taurates include those of formula (IX):

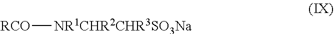

wherein R, R¹, R², and R³ are each independently selected from H or an alkyl chain having from 1-24 carbon atoms, such as from 6-20 carbon atoms, or from 8-16 carbon atoms, said chain being saturated or unsaturated, linear or branched, substituted or unsubstituted.

In various embodiments, RCO—represents the coconut acid moiety. Non-limiting examples of acyl taurate salts include sodium cocoyl taurate and sodium methyl cocoyl taurate.

### Acyl Glycinates

Non-limiting examples of useful acyl glycinates include those of formula (X):

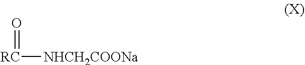

wherein R is an alkyl chain of 8 to 16 carbon atoms. Sodium is shown as the cation in the above formula (X), but the cation may be any alkali metal ion such as sodium or potassium, ammonium ions, or alkanolammonium ions such as monoethanolammonium or triethanolammonium ions.

Non-limiting examples of acyl glycinates include sodium cocoyl glycinate, sodium lauroyl glycinate, sodium myristoyl glycinate, potassium lauroyl glycinate, and potassium cocoyl glycinate, and in particular sodium cocoyl glycinate.

### Acyl Glutamates

Non-limiting examples of useful acyl glutamates include those of formula (XI):

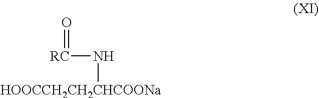

wherein R is an alkyl chain of 8 to 16 carbon atoms. Sodium is shown as the cation in the above formula (XI) but the cation may be any alkali metal ion such as sodium or potassium, ammonium ions, or alkanolammonium ions such as monoethanolammonium or triethanolammonium ions.

Non-limiting examples of acyl glutamates include dipotassium capryloyl glutamate, dipotassium undecylenoyl glutamate, disodium capryloyl glutamate, disodium cocoyl glutamate, disodium lauroyl glutamate, disodium stearoyl glutamate, disodium undecylenoyl glutamate, potassium capryloyl glutamate, potassium cocoyl glutamate, potassium lauroyl glutamate, potassium myristoyl glutamate, potassium stearoyl glutamate, potassium undecylenoyl glutamate, sodium capryloyl glutamate, sodium cocoyl glutamate, sodium lauroyl glutamate, sodium myristoyl glutamate, sodium olivoyl glutamate, sodium palmitoyl glutamate, sodium stearoyl glutamate, sodium undecylenoyl glutamate, triethanolamine mono-cocoyl glutamate, triethanolamine lauroylglutamate, and disodium cocoyl glutamate.

In an embodiment, the non-sulfate anionic surfactants of the cleansing compositions of the present disclosure may be selected from one or more acyl isethionates, their salts, or mixtures thereof.

In an embodiment, the one or more acyl isethionates, their salts, or mixtures thereof are selected from sodium cocoyl isethionate, sodium lauroyl methyl isethionate, sodium cocoyl methyl isethionate, sodium lauroyl isethionate, or mixtures thereof.

In an embodiment, the non-sulfate anionic surfactants of the cleansing compositions of the present disclosure are selected from one or more acyl sarcosinates, their salts, or mixtures thereof.

In an embodiment, the one or more acyl sarcosinates, their salts, or mixtures thereof are selected from sodium cocoyl sarcosinate, sodium lauroyl sarcosinate, potassium lauroyl sarcosinate, potassium cocoyl sarcosinate, sodium myristoyl sarcosinate, sodium oleoyl sarcosinate, sodium palmitoyl sarcosinate, ammonium lauroyl sarcosinate, or mixtures thereof.

For example, two or more, such as three or more non-sulfate anionic surfactants may be selected from acyl isethionates, acyl sarcosinates, alkyl sulfonates, alkyl sulfosuccinates, alkyl sulfoacetates, alkoxylated monoacids, and acyl amino acids such as acyl taurates, acyl glycinates, acyl glutamates, salts thereof, or mixtures thereof.

### Sulfate Anionic Surfactants

Optionally, anionic surfactants may be chosen from sulfate anionic surfactants. Such sulfate anionic surfactants may be chosen from, for example, alkyl sulfates, alkyl ether sulfates, and/or salts thereof.

By way of example, alkyl sulfates may include C₈₋₁₈ alkyl sulfates, more C₁₂₋₁₈ alkyl sulfates, such as in the form of a salt with a solubilizing cation such as sodium, potassium, ammonium, or substituted ammonium. Examples include but are not limited to sodium lauryl sulfate (SLS) and sodium dodecyl sulfate (SDS).

As a further example, alkyl ether sulfates may include those having the formula:

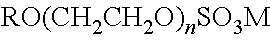

wherein:
R is an alkyl or alkenyl having from 8 to 18 (12 to 18) carbon atoms;
n is a number having an average value of greater than at least 0.5, between 1 and 3, more between 2 and 3; and
M is a solubilizing cation such as sodium, potassium, ammonium or substituted ammonium.

An example of a useful alkyl ether sulfate is sodium lauryl ether sulfate (SLES).

In some instances, useful alkyl sulfate salts and alkyl ether sulfate salts include those having the formulas (XII and XIII):

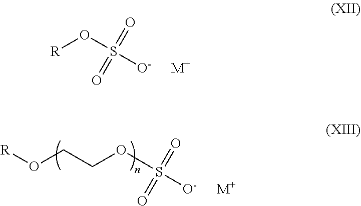

wherein:
R is an alkyl chain having from 6 to 24 carbon atoms, 8 to 18 carbon atoms, and more 12 to 18 carbon atoms;
M is a solubilizing cation such as alkali metal ions such as sodium or potassium, ammonium ions, or alkanolammonium ions such as monoethanolammonium or triethanolammonium ions; and
n is an integer from 0 to 3.

If present, the total amount of alkyl sulfates, alkyl ether sulfates, and/or salts thereof in the cleansing compositions can vary, but typically ranges from about 5 to about 50%, all amounts by weight based on the weight of the composition. In some cases, the total amount of alkyl sulfates, alkyl ether sulfates, and/or salts thereof in the cleansing composition may be from about 5 to about 45%, from about 5 to about 30%, from about 5 to about 25%, from about 5 to about 20%, from about 5 to about 15%, from about 10 to about 45%, from about 10 to about 50%, from about 10 to about 40%, from about 10 to about 35%, from about 10 to about 30%, about 10 to about 25%, about 10 to about 20%, from about 12 to about 50%, from about 12 to about 45%, from about 12 to about 40%, from about 12 to about 35%, or from about 13 to about 30%, or from about 14 to about 25%, all amounts by weight based on the weight of the composition, including ranges and sub-ranges therebetween.

In some instances, the hair treatment compositions according to the disclosure do not require silicones and/or sulfate-based anionic surfactants. Thus, any one or more (or all) of these may optionally be excluded from the cleansing compositions. In other words, the cleansing compositions may be free or essentially free of silicones and/or sulfate-based anionic surfactants. Nonetheless, in some instances, one or more silicones and/or one or more sulfate-based surfactants may optionally be included in at least certain embodiments of the cleansing compositions.

### (iv) Water

In accordance with the various embodiments, water is present in the cleansing composition in a range from about 60% to about 98%, or from about 65% to about 95%, or from about 70% to about 90%, or from about 75% to about 85%, or about 83%, or any suitable combination, sub-combination, range, or sub-range thereof by weight, based on the weight of the cleansing composition. One of ordinary skill in the art, however, will appreciate that other ranges are within the scope of the invention. Thus, water is present, by weight, based on the total weight of the cleansing composition, from about 60, 65, 70, 75, 80, 85, 90, 95 to about 98 weight percent, including increments and ranges therein and there between.

The water used may be sterile demineralized water and/or a floral water such as rose water, cornflower water, chamomile water or lime water, and/or a natural thermal or mineral water such as, for example: water from Vittel, water from the Vichy basin, water from Uriage, water from La Roche Posay, water from La Bourboule, water from Enghien-les-Bains, water from Saint Gervais-les-Bains, water from Neris-les-Bains, water from Allevar-les-Bains, water from Digne, water from Maizieres, water from Neyrac-les-Bains, water from Lons-le-saunier, water from Eaux Bonnes, water from Rochefort, water from Saint Christau, water from Les Fumades, water from Tercis-les-Bains or water from Avene. The water phase may also comprise reconstituted thermal water, that is to say a water comprising trace elements such as zinc, copper, magnesium, etc., reconstituting the characteristics of a thermal water.

The pH of the cleansing composition is not limited but is generally from 2 and 12, and in some embodiments, is one of from 3 and 11, and from 5 and 9, and from 6 and 8, and in some embodiments is 7. In some particular embodiments, the pH of the cleansing composition is in the range from about pH 4 to about pH 7. The pH can be adjusted to the desired value by addition of a base (organic or inorganic) to the cleansing composition, for example ammonia or a primary, secondary or tertiary (poly)amine, such as monoethanolamine, diethanolamine, triethanolamine, isopropanolamine or 1,3-propanediamine, or alternatively by addition of an inorganic or organic acid, advantageously a carboxylic acid, such as, for example, citric acid. In some embodiments, the pH is in the range from about pH 4 to about pH 11.

In some embodiments, the cleansing composition excludes alcohols, for example, monoalcohols such as monohydric C₁-C8 alcohols such as ethanol, propanol, butanol, isopropanol, isobutanol, and benzyl alcohol, and phenylethyl alcohol.

### Humectant

In accordance with the disclosure, one more humectants may be present in the cleansing composition. In some embodiments, the humectant may comprise one or more of polyols, including, for example, glycerin, glycerol, butylene glycol, propylene glycol/propanediol, isoprene glycol, dipropylene glycol, hexylene glycol and polyethylene glycols, monoethylene glycol, diethylene glycol, triethylene glycol, diethylene glycol, hexylene glycol, glycol ethers such as monopropylene, dipropylene and tripropylene glycol alkyl(C1-C4)ethers, squalane, triacetin, sugars, such as glucose, xylitol, maltitol, sorbitol, sucrose pentaerythritol, inositol, pyrrolidone carboxylic acid, lactic acid, lithium chloride, acetamide MEA, sodium lactate, urea, dicyanamide, hyaluronic acid, aloe vera, honey, and seaweed extract.

In some embodiments, the cleansing composition includes glycerin and sorbitol.

In accordance with the various embodiments, the amount of each humectant, when present in the cleansing composition, can range from about 0.5% to about 20% by weight, or from about 1% to about 10% by weight, or from about 3% to about 8%, or about 3.5%, or about 7%, or any suitable combination, sub-combination, range, or sub-range thereof by weight, based on the weight of the cleansing composition. One of ordinary skill in the art, however, will appreciate that other ranges are within the scope of the invention.

Thus, each of the at least one humectant in the cleansing composition, when present, is present by weight, based on the total weight of the cleansing composition, from about 0.5, 0.6, 0.7, 0.8, 0.9, 1, 1.1, 1.2, 1.3, 1.4, 1.5, 1.6, 1.7, 1.8, 1.9, 2, 2.1, 2.2, 2.3, 2.4, 2.5, 2.6, 2.7, 2.8, 2.9, 3, 3.1, 3.2, 3.3, 3.4, 3.5, 3.6, 3.7, 3.8, 3.9, 4, 4.1, 4.2, 4.3, 4.4, 4.5, 4.6, 4.7, 4.8, 4.9, 5, 6, 7, 8, 9, to about 10 percent, including increments and ranges therein and there between.

### Water-Soluble Solvents

In accordance with some embodiments, the cleansing composition may include at least one water-soluble solvent. The term “water-soluble solvent” is interchangeable with the term “water-miscible solvent” and means a compound that is liquid at 25° C. and at atmospheric pressure (760 mmHg), and it has a solubility of at least 50% in water under these conditions. In some embodiments, the water-soluble solvent has a solubility of at least 60%, 70%, 80%, or 90% in water under these conditions. Non-limiting examples of water-soluble solvents include, for example, glycerin, alcohols (for example, C1-C30, C1-C15, C1-C10, or C1-C4 alcohols), organic solvents, polyols, glycols, or combinations thereof.

As examples of organic solvents, non-limiting mentions can be made of monoalcohols and polyols such as ethyl alcohol, isopropyl alcohol, propyl alcohol, benzyl alcohol, and phenylethyl alcohol, or glycols or glycol ethers such as, for example, monomethyl, monoethyl and monobutyl ethers of ethylene glycol, propylene glycol or ethers thereof such as, for example, monomethyl ether of propylene glycol, butylene glycol, hexylene glycol, dipropylene glycol as well as alkyl ethers of diethylene glycol, for example monoethyl ether or monobutyl ether of diethylene glycol. Other suitable examples of organic solvents are ethylene glycol, propylene glycol, butylene glycol, hexylene glycol, propanediol, and glycerin. The organic solvents can be volatile or non-volatile compounds.

Further non-limiting examples of water-soluble solvents include alkanols (polyhydric alcohols such as glycols and polyols) such as glycerin, 1,2,6-hexanetriol, trimethylolpropane, ethylene glycol, propylene glycol, diethylene glycol, butylene glycol, hexylene glycol, triethylene glycol, tetraethylene glycol, pentaethylene glycol, dipropylene glycol, 1,3-butanediol, 2,3-butanediol, 1,4-butanediol, 3-methyl-1,3-butanediol, 1,5-pentanediol, tetraethylene glycol, 1,6-hexanediol, 2-methyl-2,4-pentanediol, polyethylene glycol, 1,2,4-butanetriol, 1,2,6-hexanetriol, 2-butene-1,4-diol, 2-ethyl-1,3-hexanediol, 2-methyl-2,4-pentanediol, (caprylyl glycol), 1,2-hexanediol, 1,2-pentanediol, and 4-methyl-1,2-pentanediol; alkyl alcohols having 1 to 4 carbon atoms such as ethanol, methanol, butanol, propanol, and isopropanol; glycol ethers such as ethylene glycol monomethyl ether, ethylene glycol monoethyl ether, ethylene glycol monobutyl ether, ethylene glycol monomethyl ether acetate, diethylene glycol monomethyl ether, diethylene glycol monoethyl ether, diethylene glycol mono-n-propyl ether, ethylene glycol mono-iso-propyl ether, diethylene glycol mono-iso-propyl ether, ethylene glycol mono-n-butyl ether, ethylene glycol mono-t-butyl ether, diethylene glycol mono-t-butyl ether, 1-methyl-1-methoxybutanol, propylene glycol monomethyl ether, propylene glycol monoethyl ether, propylene glycol mono-t-butyl ether, propylene glycol mono-n-propyl ether, propylene glycol mono-iso-propyl ether, dipropylene glycol monomethyl ether, dipropylene glycol monoethyl ether, dipropylene glycol mono-n-propyl ether, and dipropylene glycol mono-iso-propyl ether; 2-pyrrolidone, N-methyl-2-pyrrolidone, 1,3-dimethyl-2-imidazolidinone, formamide, acetamide, dimethyl sulfoxide, sorbit, sorbitan, acetine, diacetine, triacetine, sulfolane, or combinations thereof.

In accordance with the various embodiments the amount of the at least one water-soluble solvent, when present, is from about 0.1% to about 10%, or from about 1% to about 10%, or from about 2% to about 8%, or any suitable combination, sub-combination, range, or sub-range thereof by weight, based on the weight of the cleansing composition. One of ordinary skill in the art, however, will appreciate that other ranges are within the scope of the invention. In some embodiments, the cleansing composition includes more than one water soluble solvent, each water soluble solvent present in an amount as set forth herein above, wherein each different water soluble solvent may be present within one of the ranges selected from the ranges set forth herein above.

Thus, each one or combination of water-soluble solvents, when present, may be present by weight, based on the total weight of the cleansing composition, from about 0.1, 0.2, 0.3, 0.4, 0.5, 0.6, 0.7, 0.8, 0.9, 1, 2, 3, 4, 5, 6, 7, 8, 9, to about 10 weight percent, including increments and ranges therein and there between.

### Preservatives

In accordance with the disclosure, one or more preservatives may be present in the cleansing composition. In some embodiments, the one or more preservatives, when present, may be selected from caprylyl glycol, Myrtrimonium bromide, phenoxyethanol, hydroxyacetophenone, ethylhexyl glycerin, chlorphenesin, cetrimonium chloride, hexyl glycerin, octylglycerin, benzylglycerin, 3-heptoyl-2,2-propandiol, and 1,2-hexandiol; polyaminopropyl biguanide, also known as polyhexamethylene biguanide, or PHMB, and combinations thereof. In some embodiments, the cleansing composition is free or essentially free of one or more of caprylyl glycol, phenoxyethanol, hexyl glycerin, ethylhexylglycerin, octylglycerin, benzylglycerin, 3-heptoyl-2,2-propandiol, and 1,2-hexandiol; polyaminopropyl biguanide, also known as polyhexamethylene biguanide, or PHMB.

In some embodiments, the one more preservatives includes caprylyl glycol.

In accordance with the various embodiments, the amount of each one of the one more preservatives if present in the cleansing composition can range from about 0.01% to about 2% by weight, or from about 0.02% to about 1% by weight, or from about 0.03% to about 0.5% or any suitable combination, sub-combination, range, or sub-range thereof by weight, based on the weight of the cleansing composition. One of ordinary skill in the art, however, will appreciate that other ranges are within the scope of the invention.

Thus, each one of the one more preservatives, when present in the cleansing composition, is present by weight, based on the total weight of the cleansing composition, from about 0.01, 0.02, 0.03, 0.04, 0.05, 0.06, 0.07, 0.08, 0.09, 0.10, 0.20, 0.30, 0.40, 0.50, 0.60, 0.70, 0.80, 0.90, 1, 1.1, 1,2, 1.3, 1.4, 1.5, 1.6, 1.7, 1.8, 1.9, 2, 2.1, 2.2, 2.3, 2.4, 2.5, 2.6, 2.7, 2.8, 2.9 to about 3 weight percent, including increments and ranges therein and there between.

### Other Optional Components

In some embodiments, there may be one or more actives present in the cleansing composition, according to the disclosure, the additive selected from, for example, preservatives/anti-microbials (for example, chlorphenesin, salicylic acid, phenoxyethanol, potassium sorbate, and caprylyl glycol); actives (for example, hydroxyacetophenone); vitamins (for example, vitamin A, beta carotene, tocopherol/vitamin E, panthenol, retinol, resveratrol, vitamin C, niacinamide, derivatives thereof, and combinations thereof); hyaluronic acid in the form of hydrolyzed hyaluronic acid or sodium hyaluronate; coloring materials/pigments; essential oils; antioxidants (phenolic compounds, such as chalcones, flavones, flavanones, flavanols, flavonols, dihydroflavonols, isoflavonoids, neoflavonoids, catechins, anthocyanidins, tannins, lignans, aurones, stilbenoids, curcuminoids, alkylphenols, betacyanins, capsacinoids, hydroxybenzoketones, methoxyphenols, naphthoquinones, and phenolic terpenes, resveratrol, curcumin, pinoresinol, ferulic acid, hydroxytyrosol, cinnamic acid, caffeic acid, p-coumaric acid, baicalin (Scutellaria baicalensis root extract), pine bark extract (Pinus pinaster bark/bud extract), ellagic acid); vitamins and vitamin derivatives, such as calcium pantothenate, tocopherol and ascorbic acid; hydroxy acids; citric acid, sodium citrate, sodium chloride; neutralizing, chelating or pH-adjusting agents (for example, triethylamine (TEA), trisodium ethylenediamine disuccinate, EDTA, and sodium hydroxide); powders, fragrances, dyes, pigments; organic or mineral UV filters; and combinations thereof.

In some particular embodiments, a composition according to the disclosure may include one or a combination of additives selected from sodium hydroxide, panthenol, hydroxyacetophenone, gluconolactone, myrtrimonium bromide, sodium citrate, sodium chloride, dicaprylyl ether, citric acid, or combinations thereof.

Although the aforementioned optional components are given as an example, it will be appreciated that other optional components compatible with cosmetic applications known in the art may be used.

In accordance with the various embodiments, the amount of one or more actives and additives, alone or in combination, when present in the cleansing composition according to the disclosure can be present in a range from about 0.0001% to about 20%, and in some embodiments, from about 0.005% to about 0.01%, and in some embodiments, from about 0.01% to about 0.1%, and in some embodiments, from about 0.15% to about 5%, and in some embodiments, from about 0.40% to about 4%, and in some embodiments, from about 0.5% to about 2.5%, and in some embodiments, from about 0.1% to about 0.5% and in some embodiments, from about 1% to about 2%, or any suitable combination, sub-combination, range, or sub-range thereof by weight, based on the total weight of the cleansing composition. One of ordinary skill in the art, however, will appreciate that other ranges are within the scope of the invention.

Thus, any one or a combination of actives and additives may be present, each one or the combination present from about 0.0001, 0.0005, 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, 0.10, 0.20, 0.30, 0.40, 0.50, 0.60, 0.70, 0.80, 0.90, 1.0, 2, 3, 4, 5, 6, 7, 8, 9, 10, 11, 12, 13, 14, 15, 16, 17, 18, 19 to about 20 percent, by weight, including increments and ranges therein and there between.

In some embodiments, the cleansing composition may include one or more oils. In some examples, the oils are chosen from hydrocarbon-based oils. For example, the hydrocarbon-based oil may be a saturated hydrocarbon, an unsaturated hydrocarbon, lipids, triglycerides, a natural oil, and/or a synthetic oil. In some embodiments, the cleansing composition may include a synthetic oil selected from the group consisting of hydrogenated polyisobutene and hydrogenated polydecene. A hydrocarbon-based oil may be a non-volatile hydrocarbon-based, such as:
(i) hydrocarbon-based oils of plant origin, such as glyceride triesters, which are generally triesters of fatty acids and of glycerol, the fatty acids of which can have varied chain lengths from C4 to C24, it being possible for these chains to be saturated or unsaturated and linear or branched; these oils are in particular wheat germ oil, sunflower oil, grape seed oil, sesame oil, corn oil, apricot oil, castor oil, shea oil, avocado oil, olive oil, soybean oil, sweet almond oil, palm oil, rapeseed oil, cottonseed oil, hazelnut oil, macadamia oil, jojoba oil, alfalfa oil, poppy oil, pumpkin seed oil, marrow oil, blackcurrant oil, evening primrose oil, millet oil, barley oil, quinoa oil, rye oil, safflower oil, candlenut oil, passionflower oil, musk rose oil, and essential oils, such as Helianthus Annuus Seed Oil, Lavandula Angustifolia (lavender) Oil, Mentha Piperita Oil, Rosmarinus Officinalis (rosemary) Leaf Oil Pelargonium Graveolens flower oil, Citrus Aurantium Dulcis (orange) peel oil, Menthe Viridis (spearmint) leaf oil, Citrus Aurantifolia (lime) oil, Melaleuca Alternifolia (tea tree) leaf oil, Citrus Grandis (grapefruit) peel oil, Citrus Medica Limonum (lemon) peel oil, rose flower oil, eucalyptus globulus leaf oil, and combinations thereof.
(ii) synthetic ethers containing from 10 to 40 carbon atoms;
(iii) linear or branched hydrocarbons of mineral or synthetic origin, such as petroleum jelly, polydecenes, hydrogenated polyisobutene such as Parleam, and 40 squalane;
(iv) synthetic esters, for instance oils of formula RCOOR′ in which R represents a linear or branched fatty acid residue containing from 1 to 40 carbon atoms and R′ represents a hydrocarbon-based chain that is especially branched, containing from 1 to 40 carbon atoms on condition that R+R′ is ÿ 10, for instance Purcellin oil (cetearyl octanoate), isopropyl myristate, isopropyl palmitate, C12-C15 alkyl benzoate, such as the product sold under the trade name Finsolv TN™ or Witconol TN™ by Witco or Tegosoft TN™ by Evonik Goldschmidt, 2-ethylphenyl benzoate, such as the commercial product sold under the name X-Tend 226 by ISP, isopropyl lanolate, hexyl laurate, diisopropyl adipate, isononyl isononanoate, oleyl erucate, 2-ethylhexyl palmitate, isostearyl isostearate, diisopropyl sebacate, such as the product sold under the name of “Dub Dis” by Stearinerie Dubois, octanoates, decanoates or ricinoleates of alcohols or polyalcohols, such as propylene glycol dioctanoate; hydroxylated esters, such as isostearyl lactate or diisostearyl malate; and pentaerythritol esters; citrates or tartrates, such as di(linear C12-C13 alkyl) tartrates, such as those sold under the name Cosmacol ETI™ by Enichem Augusta Industriale, and also di(linear C14-C15 alkyl) tartrates, such as those sold under the name Cosmacol ETL™ by the same company; or acetates;
(v) fatty alcohols that are liquid at room temperature, containing a branched and/or unsaturated carbon-based chain containing from 12 to 26 carbon atoms, for instance octyldodecanol, isostearyl alcohol, oleyl alcohol, 2-hexyldecanol, 2-butyloctanol or 2-undecylpentadecanol;
(vi) higher fatty acids, such as oleic acid, linoleic acid or linolenic acid;
(vii) carbonates, such as dicaprylyl carbonate, such as the product sold under the name Cetiol CC™ by Cognis;
(viii) fatty amides, such as isopropyl N-lauroyl sarcosinate, such as the product sold under the trade name Eldew SL 205™ from Ajinomoto; and
(ix) essential oils selected from the group consisting of sunflower oil, sesame oil, peppermint oil, macadamia nut oil, tea tree oil, evening primrose oil, sage oil, rosemary oil, coriander oil, thyme oil, pimento berries oil, rose oil, anise oil, balsam oil, bergamot oil, rosewood oil, cedar oil, chamomile oil, sage oil, clary sage oil, clove oil, cypress oil, eucalyptus oil, fennel oil, sea fennel oil, frankincense oil, geranium oil, ginger oil, grapefruit oil, jasmine oil, juniper oil, lavender oil, lemon oil, lemongrass oil, lime oil, mandarin oil, marjoram oil, myrrh oil, neroli oil, orange oil, patchouli oil, pepper oil, black pepper oil, petitgrain oil, pine oil, rose otto oil, rosemary oil, sandalwood oil, spearmint oil, spikenard oil, vetiver oil, wintergreen oil, and ylang ylang.

n some embodiments the cleansing composition may include at least one branched or linear liquid alkane with carbon chain length of C11 to C20. In various embodiments, liquid alkanes may be selected from those with a carbon chain length of from C11 to C20. The liquid alkanes may be selected from those with a carbon chain length of from C11 to C20, or from C15 to C19, or one of C11, C12, C13, C14, C15, C16, C17, C18 to C19. In some particular embodiments, suitable liquid alkanes that may be used according to the disclosure include hydrocarbon-based oils containing from 8 to 16 carbon atoms, and especially branched C8-C16 alkanes such as C8-C16 isoalkanes. In some exemplary embodiments, such liquid alkanes may be chosen from isoparaffins, for instance isododecane (also known as 2,2,4,4,6-pentamethylheptane), isodecane, and isohexadecane.

The amount of each of the at least one branched or linear liquid alkane may be present in the cleansing composition in a range of from about 1% to about 35% by weight, or from about 15% to about 30% by weight, or from about 10% to about 20% or any suitable combination, sub-combination, range, or sub-range thereof by weight, based on the weight of the cleansing composition. One of ordinary skill in the art, however, will appreciate that other ranges are within the scope of the invention.

Thus, each of the at least one branched or linear liquid alkane in the cleansing composition may be present by weight, based on the total weight of the cleansing composition, from about 1, 2, 3, 4, 5, 6, 7, 8, 9, 10, 11, 12, 13, 14, 15, 16, 17, 128, 19, 20, 21, 22, 23, 24, 25, 26, 27, 28, 29, 30, 31, 32, 33, 34, to about 35 percent, including increments and ranges therein and there between.

In some embodiments the cleansing composition may include at least one emollient ester.

In some embodiments, suitable emollient esters that may be used according to the disclosure may be chosen from isopropyl myristate, isopropyl palmitate, ethylhexyl palmitate, methyl palmitate, 2-ethylhexyl palmitate, hexyl laurate, ethylhexyl laurate, isopropyl laurate, ethylhexyl oleate, ethylhexyl isononanoate, isononyl isononanoate, myristyl myristate, 2-ethylhexyl caprate/caprylate (or octyl caprate/caprylate), isostearyl neopentanoate, cetearyl ethylhexanoate, isopropyl isostearate, diisopropyl sebacate, coco caprylate/caprate, diisopropyl adipate, and combinations thereof esters of lactic acid and of fatty alcohols comprising 12 or 13 carbon atoms, and combinations thereof.

In some embodiments, the one or more emollient esters is present from about 1% to about 8% by weight, based on the weight of the cleansing composition. In some embodiments, the total amount of emollient ester does not exceed a total of about 10% by weight of the cleansing composition.

In some embodiments, each of the at least one emollient esters may be present from about 0.1% to about 15% by weight of the cleansing composition, or from about 1% to about 10% by weight, or from about 1% to about 8%, or about 1% or any suitable combination, sub-combination, range, or sub-range thereof by weight, based on the weight of the cleansing composition. When present, the total amount of emollient ester present in the cleansing composition may be in a range of from about 0.1% to about 15% by weight, or from about 1% to about 8% by weight, or any suitable combination, sub-combination, range, or sub-range thereof by weight, based on the weight of the cleansing composition. One of ordinary skill in the art, however, will appreciate that other ranges are within the scope of the invention.

Thus, each of the at least one emollient ester in the cleansing composition may be present by weight, based on the total weight of the cleansing composition, from about 0.1, 0.2, 0.3, 0.4, 0.5, 0.6, 0.7, 0.8, 0.9, 1, 2, 3, 4, 5, 6, 7, 8, 9, 10, 11, 12, 13, 14 to about 15 percent, including increments and ranges therein and there between

### EXAMPLES

### Example 1: Raw Materials

Raw Materials: Water; Rhamnolipid rhamnolipid is sold under the name Rheance One by Evoni; Rheology modified Polymers e.g., Algin and/or Anionic surfactants ex: disodium cocoyl glutamate (and) sodium; cocoyl glutamate. Other RMs include (for vars/comparatives: xanthan gum (and) sclerotium gum (and) lecithin (and) pullulan, xanthan gum, carbomer 980, hydroxypropyl starch phosphate, ammonium polyacryloyldimethyl taurate, hydroxyethyl cellulose, acrylate copolymer, sclerotium gum); caprylyl glycol, sodium hydroxide, sorbitol, tetrasodium glutamate diacetate, and glycerin.

### Example 2: Inventive and Varied Compositions

Various representative embodiments of the inventive composition are exemplified herein.

TABLE 2
Inventive and Varied Compositions

| | INV | VAR | VAR | VAR | VAR | VAR | VAR | VAR | VAR |
|---|---|---|---|---|---|---|---|---|---|
| INCI US | 1 | 1 | 2 | 3 | 4 | 5 | 6 | 7 | 8 |
| Water | 82.3 | 82.3 | 82.3 | 82.3 | 82.3 | 82.3 | 82.3 | 82.3 | 82.3 |
| Algin | 1.5 | | | | | | | | |
| XANTHAN GUM | | 1.5 | | | | | | | |
| (And) SCLEROTIUM | | | | | | | | | |
| GUM (And) | | | | | | | | | |
| LECITHIN (And) | | | | | | | | | |
| PULLULAN | | | | | | | | | |
| Xanthan Gum | | | 1.5 | | | | | | |
| Carbomer 980 | | | | 1.5 | | | | | |
| Hydropropyl Starch | | | | | 1.5 | | | | |
| Phosphate | | | | | | | | | |
| Ammonium | | | | | | 1.5 | | | |
| Polyacryloyldimethyl | | | | | | | | | |
| Taurate | | | | | | | | | |
| Hydroxyethyl | | | | | | | 1.5 | | |
| Cellulose | | | | | | | | | |
| Acrylate Copolymer | | | | | | | | 1.5 | |
| Sclerotium Gum | | | | | | | | | 1.5 |
| Caprylyl Glycol | 0.5 | 0.5 | 0.5 | 0.5 | 0.5 | 0.5 | 0.7 | 0.7 | 0.7 |
| Sodium Hydroxide | 0.1 | 0.1 | 0.1 | 0.1 | 0.1 | 0.1 | 0.1 | 0.1 | 0.1 |
| Glycolipids | 5.0 | 5.0 | 5.0 | 5.0 | 5.0 | 5.0 | 5.0 | 5.0 | 5.0 |
| (Rhamnolipid) | | | | | | | | | |
| Sorbitol | 3.5 | 3.5 | 3.5 | 3.5 | 3.5 | 3.5 | 3.5 | 3.5 | 3.5 |
| Tetrasodium | 0.1 | 0.1 | 0.1 | 0.1 | 0.1 | 0.1 | 0.1 | 0.1 | 0.1 |
| Glutamate Diacetate | | | | | | | | | |
| Glycerin | 7.0 | 7.0 | 7.0 | 7.0 | 7.0 | 7.0 | 7.0 | 7.0 | 7.0 |

### Example 3: Comparative Compositions

Various comparative benchmark compositions are exemplified herein.

TABLE 3
Reference/Comparative Compositions

| | Ref/Comp 1 | COMP 2 | COMP 3 |
|---|---|---|---|
| Ingredients | Water, Glycerin, | Aqua/Water/Eau, | Water, Glycerin, |
| | Cocamidopropyl | Cocamidopropyl | Tetrasodium EDTA, Zinc |
| | Betaine, Disodium | Hydroxysultaine, | Gluconate, Salicylic Acid, |
| | Laureth | Glycerin, Sodium Lauroyl | Sodium Laureth Sulfate, |
| | Sulfosuccinate, | Sarcosinate, Peg-150 | Capryloyl Salicylic Acid, |
| | Sodium | Pentaerythrityl | Decyl Glucoside, |
| | Cocoamphoacetate, | Tetrastearate, | Polyquaternium-47, PEG- |
| | Panthenol, | Niacinamide, Peg-6 | 150 Pentaerythrityl |
| | Niacinamide, | Caprylic/Capric | Tetrastearate (And) PEG- |
| | Pantolactone, | Glycerides, Sodium | 6 Caprylic/Capric |
| | Acrylates/C10-30 | Methyl Cocoyl Taurate, | Glycerides, Coco-Betaine, |
| | Alkyl Acrylate | Propylene Glycol, | Menthol, Sodium |
| | Crosspolymer, | Ceramide Np, Ceramide | Hydroxide, Water, |
| | Sodium Benzoate, | Ap, Ceramide Eop, | Sodium Chloride, |
| | Masking Fragrance, | Carbomer, | Hexylene Glycol, Sodium |
| | Sodium Chloride, | Methylparaben, Sodium | Chloride, Citric Acid, |
| | Citric Acid. | Chloride, Sodium Lauroyl | Sodium Hydroxide |
| | | Lactylate, Cholesterol, | |
| | | Disodium Edta, | |
| | | Propylparaben, Citric | |
| | | Acid, Tetrasodium Edta, | |
| | | Hydrolyzed Hyaluronic | |
| | | Acid, Phytosphingosine, | |
| | | Xanthan Gum | |

### Example 4: Viscosity & Stability

Compositions were tested for viscosity and stability over time. Demonstration of stability is characterized as described herein above.

TABLE 4
Viscosity & Stability Demonstration

| Stability | INV 1 | VAR 1 | VAR 2 | VAR 3 | VAR 4 | VAR 5 | VAR 6 | VAR 7 | VAR 8 |
|---|---|---|---|---|---|---|---|---|---|
| Viscosity (M3) | 44.3 | 32.5 | 31.4 | 22.7 | <10 | <10 | 15.8 | <10 | 27.9 |
| at 25° C. | | | | | | | | | |
| Stability 25° C. | Stable | Stable | Fail | Fail | Fail | Fail | Fail | Fail | Stable |
| | 2 mos. | 2 mos. | | | | | | | 2 mos. |
| Stability 45° C. | Stable | Stable | Fail | Fail | Fail | Fail | Fail | Fail | Stable |
| | 2 mos. | 2 mos. | | | | | | | 2 mos. |

Referring now to Table 4, the results of this study show that in some embodiments, the selected thickening polymer can affect stability and that ideal stability was observed with inventive compositions INV 1, VAR 1 and VAR 8, wherein these compositions did not demonstrate appreciable change in viscosity, or formation of phase break, precipitates, clouding, or crystallization.

### Example 5: Cleanability/Pore Cleansing (Bioskin Substrate)

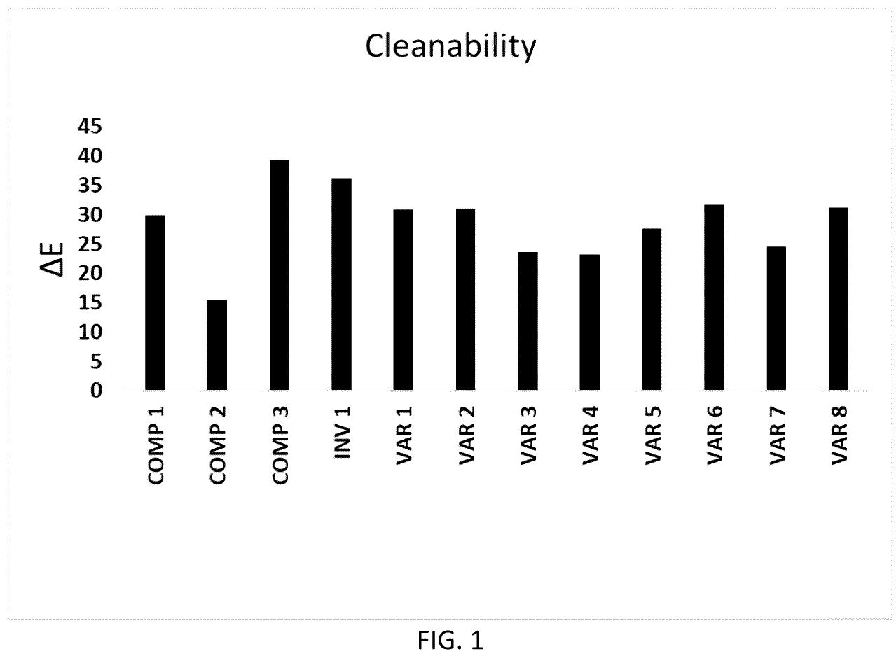

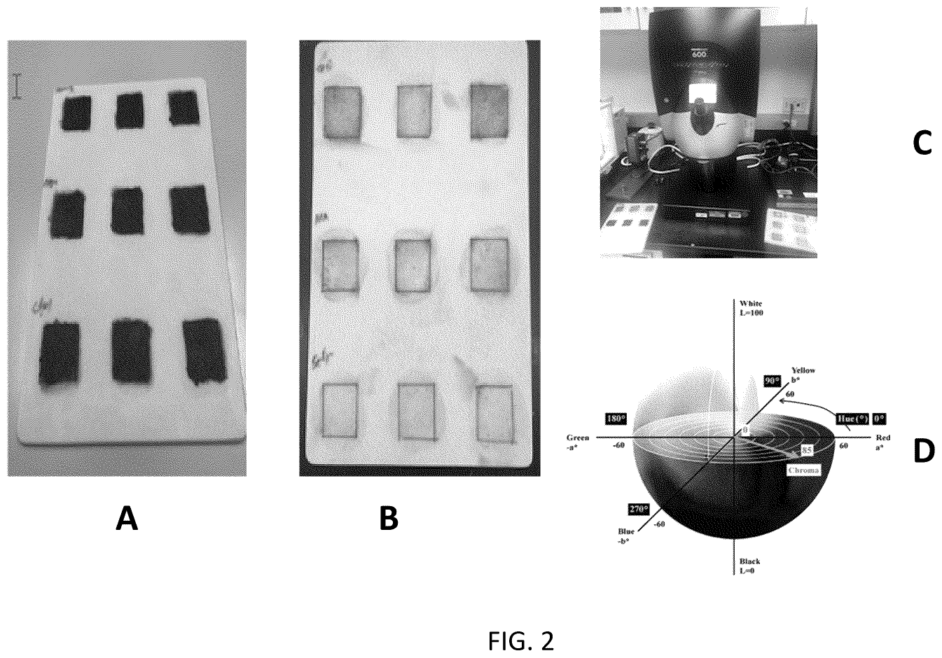

The inventive, var and reference/comparative compositions were evaluated for cleanability performance. Results are shown in Table 5 and FIG. 1 and FIG. 2.

### Cleanability Test Protocol:

Pollutant: synthetic pollution material (sebollution); Instrument: Datacolor™ 600 spectrophotometer, force controller; Procedure: Evenly spread 10 mg sebollution in 1 inch by 1 inch square area on bioskin; Air dry the painted area overnight; Measure color degree of the painted area by Datacolor 600 spectrophotometer; Add 2 parts water to 1 part product, make sure the blend is well mixed; Dose 13 mL mixture solution onto each painted area in bioskin; Rub the painted area 30 circles by fingertip and control force applied between scale 6-8 using force controller (L'Oreal SWIFT); Rinse the rubbed area under water faucet at flow rate 17 gallon/hr, temperature 37 Celsius degree; Use Kim wipe gently absorb the extra water on the rinsed bioskin; Measure color degree of the washed area by Datacolor 600 spectrophotometer; Cleanability is calculated as the color difference (ΔE) before and after rinse.

According to the disclosure, a score of ΔE>30-45 demonstrates that the cleansing removes surface pollutant/makeup/debris and also provides deep cleaning as evidenced by the increase in ΔE, suggesting deep cleansing into pores as observed at least in the in vitro artificial skin substrates.

TABLE 5
Cleanability/Pore Cleansing

| Formulation | ΔE |
|---|---|
| COMP 1 | 46.06 |
| COMP 6 | 68.68 |
| COMP 4 | 26.85 |
| COMP 1 | 65.71 |
| INV 1 | 63.24 |
| INV 2 | 58.08 |
| COMP 3 | 69.37 |
| INV 3 | 63.86 |
| INV 4 | 40.42 |
| COMP 5 | 82.26 |

Cleanability Performance: Referring to Table 5 and FIG. 1-2, it is evident that the inventive composition demonstrates at least comparable (with respect to COMP 3) or improved deep cleaning and removability performance as compared with benchmark comparative compositions and this cleaning is accomplished in the absence of conventional and often more harsh surfactant or less desirable/less natural ingredients.

### Example 6: Removability (Foundation and Lipstick)

Evaluation of Longwear Foundation and Lipstick on Hand/Arm: Substrate: Human Hand or Arm; Preparation: Apply 20 μl of foundation in a 3×3 inch square on Hand or Arm SURFACE and spread evenly within the square. Air dry at room temp for 30 min; Equipment: Carma, taking pictures (before/after).

Method for removal: Drop onto substrate tested formula about 1 g formulation and 1 g water. Using wipe material, slowly wipe off the surface in 20 wipe cycles.

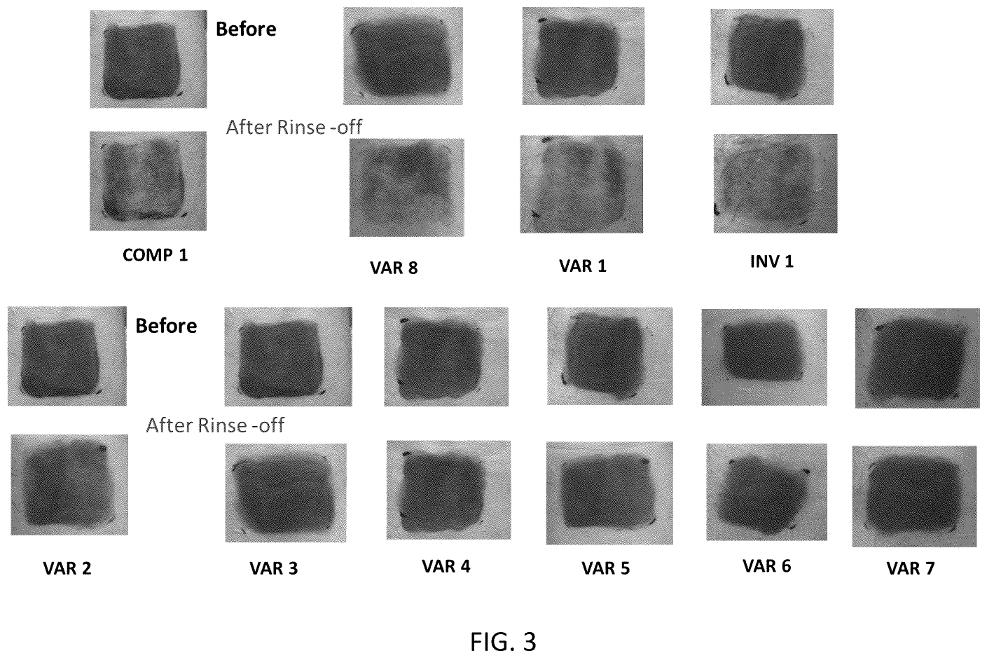

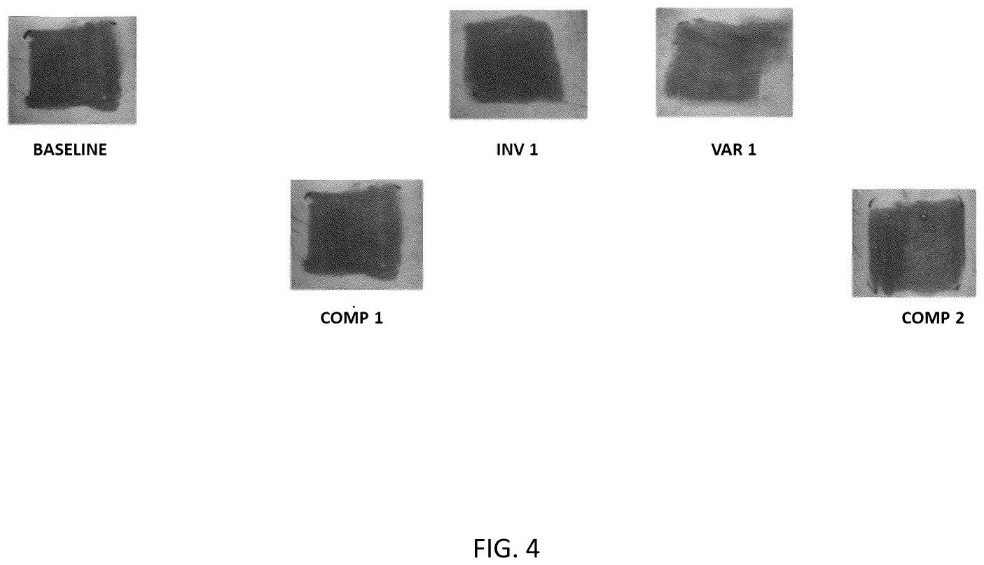

The inventive, var and reference/comparative compositions were evaluated for removability performance. Results are shown in Table 6 and FIG. 3 and FIG. 4.

TABLE 6
Removability

| Formulation | Foundation | Lipstick |
|---|---|---|
| INV 1 | 8 | 2 |
| VAR 1 | 9 | 3 |
| VAR 2 | 5 | |
| VAR 3 | 3 | |
| VAR 4 | 3 | |
| VAR 5 | 3 | |
| VAR 6 | 2 | |
| VAR 7 | 1 | |
| VAR 8 | 8 | |
| COMP 1 | 7 | 1 |
| COMP 2 | | 3 |
| COMP 3 | 10 | 5 |
| COMP 4 | | 4 |

KEY: Foundation Removal: max (10) to min (1); Lipstick Removal: max (5) to min (1)

Referring now to Table 6 and FIG. 3 and FIG. 4, the results of this study show that the exemplified embodiments of the inventive composition provide comparable or enhanced foundation and lipstick removal as compared with benchmark/comparative compositions.

### Example 7: In Vitro Removability of Waterproof Regular Wear Mascara (not Long-Wear)

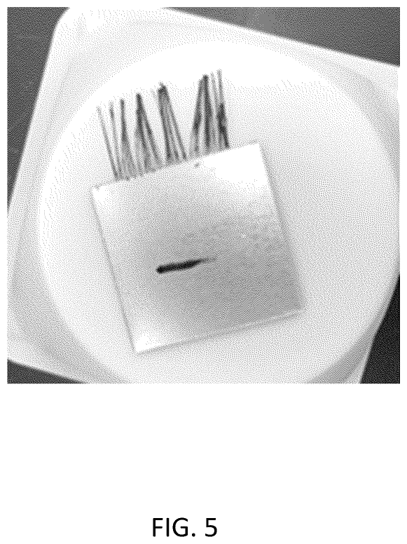

Protocol: Substrate: Fake eyelash (shown in FIG. 5); Mascara: L'Oreal Feline Voluminous; Preparation: Weigh substrate (before/after). Apply 30 stroke product on fake eyelash substrate; Air dry at room temp for 24 hrs; Method for removal: Drop onto substrate tested formula about 1 g formulation and 1 g water. Using wipe material, slowly wipe off the surface in 20 wipe cycles. Dry, then weigh.

### Makeup (Mascara) Removal:

% Removed=wt of mascara removed(based on weight of substrate after cleansing)/wt of mascara applied(based on weight of substrate after application)×100

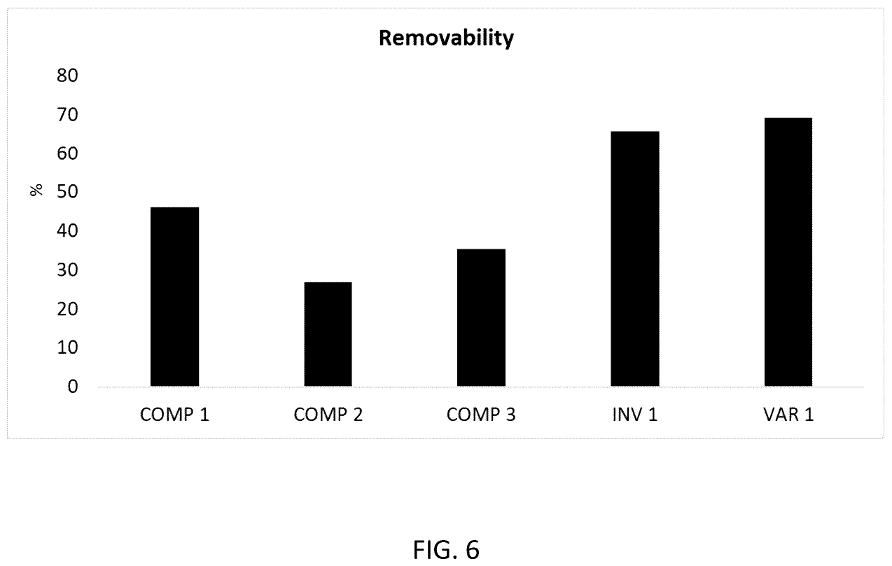

The inventive, var and reference/comparative compositions were evaluated for removability performance. Results are shown in Table 7 and FIG. 6.

TABLE 7
Removability (waterproof regular wear mascara (not long-wear))

| FORMULATION | % Removed |
|---|---|
| COMP 1 | 46.06 |
| COMP 2 | 26.85 |
| COMP 3 | 35.37 |
| INV 1 | 65.71 |
| VAR 1 | 69.37 |

Removability Performance: Referring to Table 7 and FIG. 6, it is evident that the inventive composition demonstrates improved removal performance as compared with benchmark comparative compositions.

### Example 8: Foaming

The inventive, var and reference/comparative compositions were evaluated for foaming performance. Results are shown in Table 8.

TABLE 8
Foaming

| | Foaming |
|---|---|
| | (INCLUDES |
| Formulation | *Rhamnolipid) |
| COMP 1 | 3 |
| COMP 2 | 3.5 |
| INV 1 | 6 |
| VAR 1 | 6.5 |
| VAR 2 | 2 |
| VAR 3 | 1 |
| VAR 4 | 2 |
| VAR 5 | 2 |
| VAR 6 | 1 |
| VAR 7 | 3 |
| VAR 8 | 2 |

Key: max foam (10) to min foam (1)

Foaming Performance: Referring to Table 8, it is evident that embodiments of the inventive composition demonstrate improved foaming performance as compared with benchmark comparative compositions.

### Example 9: Foaming Analysis

Foam formation with the inventive and comparative compositions was evaluated using standard foam analysis procedure.

Instrument: Kruss DFA100 Dynamic Foam Analyzer bubble count/mm2 (data use for foaming property, bigger=foaming better)

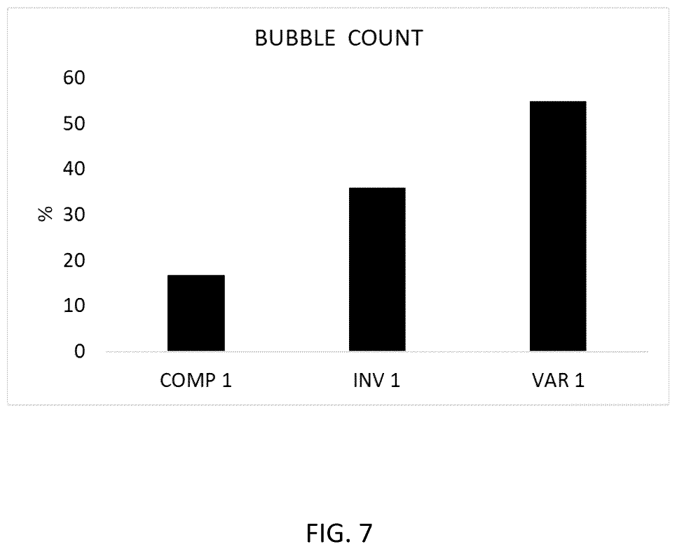

The inventive and reference/comparative compositions were evaluated for foam bubble count (foam volume). Results are shown in Table 9 and FIG. 7.

TABLE 9
Foam Analysis

| Formulation | BUBBLE COUNT |
|---|---|
| COMP 1 | 16.8 |
| INV 1 | 36 |
| VAR 1 | 54.91 |

Foaming Analysis Performance: Referring now to FIG. 7 and Table 9, the results of this study show that the tested embodiments INV 1 and VAR 1 of the inventive composition perform much better than the comparative cleansing product in terms of bubble formation per unit area.

While the disclosure has been described with reference to an embodiment, it will be understood by those skilled in the art that various changes may be made and equivalents may be substituted for elements thereof without departing from the scope of the disclosure. In addition, many modifications may be made to adapt a particular situation or material to the teachings of the disclosure without departing from the essential scope thereof. Therefore, it is intended that the disclosure not be limited to the particular embodiment disclosed as the best mode contemplated for carrying out this disclosure, but that the disclosure will include all embodiments falling within the scope of the appended claims.

The articles “a” and “an,” as used herein, mean one or more when applied to any feature in embodiments of the present disclosure described in the specification and claims. The use of “a” and “an” does not limit the meaning to a single feature unless such a limit is specifically stated. The article “the” preceding singular or plural nouns or noun phrases denotes a particular specified feature or particular specified features and may have a singular or plural connotation depending upon the context in which it is used. The adjective “any” means one, some, or all indiscriminately of whatever quantity.

“At least one,” as used herein, means one or more and thus includes individual components as well as mixtures/combinations.

The transitional terms “comprising,” “consisting essentially of” and “consisting of”, when used in the appended claims, in original and amended form, define the claim scope with respect to what unrecited additional claim elements or steps, if any, are excluded from the scope of the claim(s). The term “comprising” is intended to be inclusive or open-ended and does not exclude any additional, unrecited element, method, step or material. The term “consisting of” excludes any element, step or material other than those specified in the claim and, in the latter instance, impurities ordinarily associated with the specified material(s). The term “consisting essentially of” limits the scope of a claim to the specified elements, steps or material(s) and those that do not materially affect the basic and novel characteristic(s) of the claimed disclosure. All materials and methods described herein that embody the present disclosure can, in alternate embodiments, be more specifically defined by any of the transitional terms “comprising,” “consisting essentially of,” and “consisting of.”

In some embodiments, the terms “Exclude,” “Free” and “Essentially Free” mean that no reliably measurable excluded material, typically 0% by weight, based on the total weight of the cleansing composition, or, for example, no amount greater than about 5%, by weight, based on the total weight of the cleansing composition, of any excluded material as described herein, is present in the cleansing composition. The terms “Exclude,” “Free” and “Essentially Free” mean that, while it is preferred that no excluded material is present in the cleansing composition, it is possible to have very small amounts of the excluded material in the cleansing composition of the invention, provided that these amounts do not materially affect the advantageous properties of the cleansing composition. In particular, the terms “Exclude,” “Free” and “Essentially Free” mean that material can be present in the cleansing composition at an amount of less than 5%, or less than 4%, or less than 3%, or less than 2%, or less than 1%, or less than 0.1% by weight, based on the total weight of the cleansing composition. In accordance with the various embodiments, it is contemplated that some or all of following ingredients may be excluded from the cleansing composition according to the invention: alcohols, for example but not limited to, monohydric alcohols; powders comprising Nylon-12, polymethyl methacrylate, polyethylene beads, and powders which are considered microplastics; lactic acid, glycolic acid, tartaric acid, mandelic acid, citric acid, salicylic acid and derivatives thereof (including 5-n-octanoylsalicylic acid, salicylate, sodium salicylate, and willow extract), capryloyl salicylic acid, beta hydroxybutanoic acid, propionic acid, beta-hydroxy beta-methylbutyric acid, carnitine tropic acid, and trethocanic acid; alkyl polyglucosides selected from the group consisting of decyl glucoside, lauryl glucoside, octyl glucoside, coco glucoside, caprylyl/capryl glucoside, and sodium lauryl glucose carboxylate, and combinations thereof; surfactants with a C10-C20 fatty alcohol or acid hydrophobe condensed with from about 2 to about 100 moles of ethylene oxide or propylene oxide per mole of hydrophobe; C2-C10 alkyl phenols condensed with from 2 to 20 moles of alkylene oxide; mono- and di-fatty acid esters of ethylene glycol; fatty acid monoglyceride; sorbitan; mono- and di- C8-C20 fatty acids; polyoxyethylene sorbitan; alkyl polyglycosides and saccharide fatty amides (e.g. methyl gluconamides); alkyl ether sulfate and sulfonates; alkyl sulfates and sulfonates; alkylbenzene sulfonates; alkyl and dialkyl sulfosuccinates; C8-C20 acyl isethionates; C8-C20 alkyl ether phosphates; alkylethercarboxylate, PEG-100 Stearate; PEG-20 Stearate and other esters of Poly(Ethylene Glycol); polyethylene glycol (PEG) surfactants, polypropylene glycol (PPG) surfactants, sulfate surfactants; taurates, for example, taurate surfactants; Sucrose Stearate and other emulsifiers based on sugar esters; Glyceryl Stearate and other glycerol esters; Disodium Ethylene Dicocamide PEG-15 Disulfate; Sodium Steroyl Glutamate and other fatty amides; Steareth-100 and other fatty ethers, cationic surfactants, and other cationic compounds; silicones, such as silicone polymers, silicone elastomers, and silicone oils, for example selected from dimethicone, cyclopentasiloxane, cyclohexasiloxane, and combinations thereof; phthalates, parabens, sulfates, polyquaternium, microplastics, synthetic dyes, gelling agents, and combinations thereof.

Other than in the operating examples, or where otherwise indicated, all numbers expressing quantities of ingredients and/or reaction conditions are to be understood as being modified in all instances by the term “about,” meaning within 10% of the indicated number (e.g., “about 10%” means 9%-11% and “about 2%” means 1.8%-2.2%).

All percentages and ratios are calculated by weight unless otherwise indicated. All percentages are calculated based on the total composition unless otherwise indicated. Generally, unless otherwise expressly stated herein, “weight” or “amount” as used herein with respect to the percent amount of an ingredient refers to the amount of the raw material comprising the ingredient, wherein the raw material may be described herein to comprise less than and up to 100% activity of the ingredient. Therefore, weight percent of an active in a composition is represented as the amount of raw material containing the active that is used, and may or may not reflect the final percentage of the active, wherein the final percentage of the active is dependent on the weight percent of active in the raw material.

All ranges and amounts given herein are intended to include subranges and amounts using any disclosed point as an end point. Thus, a range of “1% to 10%, such as 2% to 8%, such as 3% to 5%,” is intended to encompass ranges of “1% to 8%,” “1% to 5%,” “2% to 10%,” and so on. All numbers, amounts, ranges, etc., are intended to be modified by the term “about,” whether or not so expressly stated. Similarly, a range given of “about 1% to 10%” is intended to have the term “about” modifying both the 1% and the 10% endpoints. Further, it is understood that when an amount of a component is given, it is intended to signify the amount of the active material unless otherwise specifically stated.

Notwithstanding that the numerical ranges and parameters setting forth the broad scope of the disclosure are approximations, unless otherwise indicated the numerical values set forth in the specific examples are reported as precisely as possible. Any numerical value, however, inherently contains certain errors necessarily resulting from the standard deviation found in their respective testing measurements. The example that follows serves to illustrate embodiments of the present disclosure without, however, being limiting in nature.

All publications and patent applications cited in this specification are herein incorporated by reference, and for any and all purposes, as if each individual publication or patent application were specifically and individually indicated to be incorporated by reference. In the event of an inconsistency between the present disclosure and any publications or patent application incorporated herein by reference, the present disclosure controls.

1. A cleansing composition, comprising: at least one glycolipid comprising at least one rhamnolipid; at least one nature-based viscosity modifying polymeric thickener for a water-based system, or at least one anionic surfactant, or a combination thereof; and water present from about 60% to about 98% by weight of the cleansing composition, the foaming cleansing composition excluding chitosan and any other cationic polymer.

2. The cleansing composition according to claim 1, wherein the at least one glycoprotein comprising rhamnolipid is sold under the name Rheance One by Evonik.

3. The cleansing composition according to claim 1, comprising at least one nature-based viscosity modifying polymeric thickener for a water-based system, selected from the group consisting of algin, xanthan gum (and) sclerotium gum (and) lecithin (and) pullulan (Siligel™), xanthan gum, carbomer 980, hydroxypropyl starch phosphate, ammonium polyacryloyldimethyl taurate, hydroxyethyl cellulose, acrylate copolymer, sclerotium gum, and combinations thereof.

4. The cleansing composition according to claim 1, comprising at least one nature-based viscosity modifying polymeric thickener for a water-based system, selected from algin, xanthan gum (and) sclerotium gum (and) lecithin (and) pullulan (Siligel™) or a combination thereof, and excluding any one or more of xanthan gum, carbomer 980, hydroxypropyl starch phosphate, ammonium polyacryloyldimethyl taurate, hydroxyethyl cellulose, acrylate copolymer, sclerotium gum, or combinations thereof.

5. The cleansing composition according to claim 1, comprising at least one anionic surfactant.

6. The cleansing composition according to claim 1, comprising at least one anionic surfactant comprising disodium cocoyl glutamate (and) sodium cocoyl glutamate.

7. The cleansing composition according to claim 1, comprising one or more of humectants, antimicrobial agents, pH adjusters, chelating agents, skin care actives, or combinations thereof, and wherein the composition is free or essentially free of one or more of silicones, alcohol, paraben, fragrance, sulfates, or combinations thereof.

8. The cleansing composition according to claim 1, comprising one or more of glycerin, sorbitol, caprylyl glycol, sodium hydroxide, tetrasodium glutamate diacetate, or combinations thereof.

9. The cleansing composition according to claim 1, wherein the rhamnolipid is present in the cleansing composition from about 0.1% to about 20% by weight of the cleansing composition.

10. The cleansing composition according to claim 1, comprising at least one nature-based viscosity modifying polymeric thickener for a water-based system present from about 0.1% to about 20% by weight, based on the weight of the cleansing composition.

11. The cleansing composition according to claim 1, wherein the rhamnolipid is present in the cleansing composition at about 5% and comprising at least one nature-based viscosity modifying polymeric thickener for a water-based system present at about 1.5% and is selected from the group consisting of algin, xanthan gum (and) sclerotium gum (and) lecithin (and) pullulan (Siligel™), xanthan gum, carbomer 980, hydroxypropyl starch phosphate, ammonium polyacryloyldimethyl taurate, hydroxyethyl cellulose, acrylate copolymer, sclerotium gum, and combinations thereof, all amounts by weight of the cleansing composition.

12. The cleansing composition according to claim 6, wherein the at least one anionic surfactant in the cleansing composition is present from about 0.01% to about 1%, by weight of the composition.

13. The cleansing composition according to claim 1, comprising at least one humectant, present in the cleansing composition from about 1% to about 10%, and wherein water is present in the cleansing composition in a range from about 75% to about 85%, all amounts by weight of the composition.

14. A cleansing composition, comprising:
a. at least one glycolipid comprising at least one rhamnolipid present in the cleansing composition from about 0.1% to about 20% by weight of the cleansing composition;
b. (i) at least one nature-based viscosity modifying polymeric thickener for a water-based system present in the cleansing composition from about 0.1% to about 20% by weight of the cleansing composition, or (ii) at least one anionic surfactant present from about 0.01% to about 1%, the nature-based viscosity modifying polymeric thickener for a water-based system selected from the group consisting of algin, xanthan gum (and) sclerotium gum (and) lecithin (and) pullulan (Siligel™), xanthan gum, carbomer 980, hydroxypropyl starch phosphate, ammonium polyacryloyldimethyl taurate, hydroxyethyl cellulose, acrylate copolymer, sclerotium gum, and combinations thereof;
c. at least one humectant present from about 1% to about 15% by weight of the cleansing composition; and
d. water present from about 60% to about 98% by weight of the cleansing composition
wherein the composition is a makeup remover
the foaming cleansing composition excluding chitosan and any other cationic polymer.

15. The cleansing composition according to claim 14, comprising a nature-based viscosity modifying polymeric thickener for a water-based system the group consisting of comprising algin, xanthan gum (and) sclerotium gum (and) lecithin (and) pullulan (Siligel™) or a combination thereof, and excluding any one or more of xanthan gum, carbomer 980, hydroxypropyl starch phosphate, ammonium polyacryloyldimethyl taurate, hydroxyethyl cellulose, acrylate copolymer, sclerotium gum, and combinations thereof.

16. The cleansing composition according to claim 14, wherein the rhamnolipid is present in the cleansing composition at about 5% and wherein the at least one polymeric thickener for a water-based system is present at about 1.5% is selected from the group consisting of algin, xanthan gum (and) sclerotium gum (and) lecithin (and) pullulan (Siligel™), xanthan gum, carbomer 980, hydroxypropyl starch phosphate, ammonium polyacryloyldimethyl taurate, hydroxyethyl cellulose, acrylate copolymer, sclerotium gum, and combinations thereof, and wherein the at least one humectant comprises glycerin and sorbitol present in combination in an amount that is about 10.5%, all amounts by weight of the cleansing composition.

17. The cleansing composition according to claim 14, comprising one or more of antimicrobial agents, pH adjusters, chelating agents, skin care actives, or combinations thereof, and wherein the composition is free or essentially free of one or more of silicones, alcohol, paraben, fragrance, sulfates, or combinations thereof, and wherein water is present in the cleansing composition in a range from about 75% to about 85%, by weight of the composition.

18. A method for deep cleansing skin comprising:
a. Providing a cleansing composition according to claim 1;
b. Applying the composition to skin by rubbing to produce foaming, followed by rubbing to remove makeup from at least the surface of the skin;
c. Rinsing with water to remove substantially all of the cleansing composition.

19. The method according to claim 18, wherein:
a. the at least one glycolipid comprising at least one rhamnolipid is present in the cleansing composition from about 0.1% to about 20% by weight of the cleansing composition;
b. the composition comprises at least one nature-based viscosity modifying polymeric thickener for a water-based system present in the cleansing composition from about 0.1% to about 20% by weight of the cleansing composition and selected from algin, xanthan gum (and) sclerotium gum (and) lecithin (and) pullulan (Siligel™), xanthan gum, carbomer 980, hydroxypropyl starch phosphate, ammonium polyacryloyldimethyl taurate, hydroxyethyl cellulose, acrylate copolymer, sclerotium gum, or combinations thereof;
c. the composition comprises at least one humectant present from about 1% to about 15% by weight of the cleansing composition; and
d. water is present from about 60% to about 98% by weight of the cleansing composition.

20. The method according to claim 19, wherein the rhamnolipid is present in the cleansing composition at about 5% and wherein the at least one polymeric thickener for a water-based system is present at about 1.5% is selected from the group consisting of algin, xanthan gum (and) sclerotium gum (and) lecithin (and) pullulan (Siligel™), xanthan gum, carbomer 980, hydroxypropyl starch phosphate, ammonium polyacrylovldimethyl taurate, hydroxyethyl cellulose, acrylate copolymer, sclerotium gum, and combinations thereof, and wherein the humectant comprises glycerin and sorbitol present in combination in an amount that is about 10.5%, all amounts by weight of the cleansing composition.
